(19) United States
(12) Patent Application Publication
(10) Pub. No.: US 2026/0193713 A1
(43) Pub. Date: Jul. 9, 2026

(54) Detecting Liver Cancer Using Cell-Free DNA Fragmentation

(71) Applicant: The Johns Hopkins University, Baltimore, MD (US)

(72) Inventors: Victor E. Velculescu, Glenwood, MD (US); Zachariah Foda, Elkton, MD (US); Akshaya Annapragada, Baltimore, MD (US); Daniel C. Bruhm, Baltimore, MD (US); Robert B. Scharpf, Baltimore, MD (US)

(21) Appl. No.: 19/127,685

(22) PCT Filed: Nov. 6, 2023
(86) PCT No.: PCT/US2023/078857

§ 371 Date: May 6, 2025

Related U.S. Application Data
(60) Provisional application No. 63/423,003, filed on Nov. 6, 2022.

(51) Int. Cl. C12Q 1/6886; C12Q 1/6804; G16B 30/20; G16B 40/20

(52) U.S. Cl. CPC C12Q 1/6886; C12Q 1/6804; G16B 30/20; G16B 40/20

(57) ABSTRACT

In one aspect, methods for liver cancer detection are provided that use a combination of genome-wide mutation and fragmentation features of cell free DNA (cfDNA) that facilitate cancer screening. In one aspect, a method of diagnosing liver diseases or disorders in a subject, comprises isolating circulating cfDNA from a subject; conducting whole genome sequencing of cfDNA molecules to generate genomic libraries and fragmentation profiles; comparing the fragmentation profiles to healthy subjects and/or reference genome, and diagnosing whether the subject has a liver disease or disorder.

The present application claims the benefit of priority of U.S. provisional application No. 63/423,003 filed Nov. 6, 2022, which is incorporated herein by referenced in its entirety.

### STATEMENT REGARDING FEDERALLY SPONSORED RESEARCH

This invention was made with government support under grants GM136577, CA121113, CA006973, and CA233259 awarded by the National Institutes of Health. The government has certain rights in the invention.

### BACKGROUND

Liver cancer is a cause of a staggering amount of morbidity and mortality worldwide, with more than 900 thousand newly diagnosed cases each year and more than 800 thousand deaths (1). In the United States, liver cancer is one of the few cancers that has shown an increase in incidence and mortality over the last 20 years. Ninety percent of cases of liver cancer are hepatocellular carcinoma (HCC) and survival is highly dependent on the stage of the disease at diagnosis. The five-year survival is 34% when the cancer is localized (44% of patients), 12% when regional (27% of patients), and 3% when distant disease is found (18% of patients) (2). There is a large, well-defined population that is at significantly increased risk for HCC, including individuals with chronic hepatitis B (HBV) infection or with cirrhosis from various causes including hepatitis C (HCV) (3), non-alcoholic fatty liver disease (NAFLD) (4), heavy alcohol use (5), aflatoxin, and other conditions (6). Worldwide, there are 350 million individuals with chronic viral hepatitis infection and 50 million with cirrhosis (7). In the US, 4.5 million individuals have chronic HCV, and 29 million have been diagnosed with NAFLD. Up to one third of those with cirrhosis and between 25-40% with HBV will develop HCC over their lifetime, with an up to 8% annual risk for patients with cirrhosis (8). A growing group of individuals at risk for liver cancer, including 29 million in the US, have NAFLD and 20% of the HCC that develops in this population occurs without cirrhosis (9). Medical societies throughout the world recommend screening for the highest risk populations, currently with abdominal ultrasound imaging with or without alpha-fetoprotein (AFP). Overall adherence to international guidelines, however, remains low, with less than 1 in 5 eligible individuals worldwide receiving some level of surveillance and less than 2% following recommended screening (10-12). Many factors contribute to low adherence to screening guidelines, including identification of high-risk individuals, the requirement of infrastructure and personnel needed for imaging-based screening methods (11). Current screening tests that include ultrasound imaging, with or without AFP, have shown limited sensitivity varying from 47-84% with specificities from 67% to over 90% (13). Additionally, the lack of non-invasive diagnostic approaches for NAFLD suggests that the population not currently covered by HCC screening recommendations is increasing. Therefore, there is a great need for development of accessible and sensitive screening approaches for HCC worldwide.

One recent avenue for overcoming these challenges has been the development of novel blood-based cell free DNA (cfDNA) biomarkers for the detection of cancer. Somatic mutation-based approaches have been used as biomarkers for liver cancer but are limited by the need for tissue-based mutation identification, and by the few changes detectable in plasma (14). Methylation profiling, both at specific sites and throughout the whole genome, as well as copy number changes, have also provided feasible avenues for detection of liver cancer but their detection sensitivities in very early-stage disease remain suboptimal (15-20). Recently developed multi-cancer early detection tests appear useful for detection of many cancers (including liver cancer) in an average risk cohort (21), but there are no published reports of using these approaches in a population at high-risk of HCC. Additionally, the cost of most cfDNA-based tests are much higher than estimates of what would be affordable for screening tests in the US and world-wide (22). Combining these approaches with AFP has increased performance, but requires two separate tests and still has limitations in early stage disease (23).

### SUMMARY

Provided herein is a non-invasive and an ultrasensitive analysis of single cell-free DNA (cfDNA) molecules to detect changes in the fragmentation profiles across the genome. Patients with liver cancer were found to have altered fragmentation profiles as a result of genomic and chromatin changes in liver cancer, including from regions associated with liver-specific transcription factors as compared to healthy subjects.

Accordingly, in one aspect, a method of diagnosing liver diseases or disorders in a subject, comprises isolating circulating cell free DNA (cfDNA) from a subject; conducting whole genome sequencing of cfDNA molecules to generate genomic libraries and fragmentation profiles; comparing the fragmentation profiles to healthy subjects and/or reference genome, and diagnosing whether the subject has a liver disease or disorder. In certain embodiments, the fragmentation profiles are consistent for subjects without cancer whereas the fragmentation profiles are highly variable for subjects with liver cancer. In certain embodiments, the method further comprises identifying cellular origins of the cfDNA fragmentation profiles, wherein the identification of the cellular origins of cfDNA fragmentation profiles comprises comparing genome-wide fragmentome profiles to high-throughput sequencing chromosome conformation capture (Hi-C). In certain embodiments, the cellular origins of cfDNA fragmentation profiles of healthy subjects correlate to lymphoblastoid cells as the cellular origin. In certain embodiments, the cellular origins of cfDNA fragmentation profiles of subjects with liver cancer correlate to cfDNA fragmentation profiles of chromatin compartments of peripheral blood cells. In certain embodiments, the method further comprises determining whether cfDNA fragmentation profiles correlate with altered DNA binding of transcription factors. In certain embodiments, the transcription factor DNA binding sites are determined by calculating aggregate cfDNA coverage across all identified transcription factor DNA binding sites compared to overall adjacent genomic coverage to produce a single metric for each transcription factor per sample. In certain embodiments, the transcription factor DNA binding sites identified in subjects with liver cancer are compared to transcription factor DNA binding sites in healthy subjects. In certain embodiments, the cfDNA fragmentation profiles of subjects with liver cancer correlate with altered DNA binding of transcription factors. In certain embodiments, the method further comprises determining chromosomal gains or losses in liver cancer subjects as compared to healthy subjects. In certain embodiments, the method further comprises a machine learning model for determining changes in cfDNA fragmentation profiles that classifies the subject as a cancer patient based on the cfDNA fragmentation profile for the subject. In certain embodiments, the machine learning model generates a score for each subject based on a combination of regional and large-scale fragmentation profiles. In certain embodiments, the generated score is diagnostic of liver cancer stages.

In another aspect, systems and methods are disclosed for detecting cancer by conducting whole genome sequencing of cfDNA molecules to generate genomic libraries and fragmentation profiles and the data entered into computer memory; executing a machine learning model for determining changes in cfDNA fragmentation profiles that classifies the subject as a cancer patient based on the cfDNA fragmentation profile for the subject.

In another aspect, a method of diagnosing liver cancer, comprises isolating circulating cell free DNA (cfDNA) from a biological sample and conducting whole genome sequencing of cfDNA molecules to generate genomic libraries and fragmentation profiles; identifying cellular origins of the cfDNA fragmentation profiles comprising comparing genome-wide fragmentome profiles to high-throughput sequencing chromosome conformation capture (Hi-C); correlating cfDNA fragmentation profiles with altered DNA binding of transcription factors; determining chromosomal gains or losses in liver cancer subjects as compared to healthy subjects; executing a machine learning model for determining changes in cfDNA fragmentation profiles that classifies the subject as a cancer patient based on the cfDNA fragmentation profile for the subject; thereby, diagnosing liver cancer and administering a cancer treatment to the subject. In certain embodiments, the cellular origins of cfDNA fragmentation profiles of healthy subjects correlate to lymphoblastoid cells as the cellular origin. In certain embodiments, the cellular origins of cfDNA fragmentation profiles of subjects with liver cancer correlate to cfDNA fragmentation profiles of chromatin compartments of peripheral blood cells. In certain embodiments, the transcription factor DNA binding sites are determined by calculating aggregate cfDNA coverage across all identified transcription factor DNA binding sites compared to overall adjacent genomic coverage to produce a single metric for each transcription factor per sample. In certain embodiments, the transcription factor DNA binding sites identified in subjects with liver cancer are compared to transcription factor DNA binding sites in healthy subjects. In certain embodiments, the cfDNA fragmentation profiles of subjects with liver cancer correlate with altered DNA binding of transcription factors. In certain embodiments, the machine learning model generates a score for each subject based on a combination of regional and large-scale fragmentation profiles. In certain embodiments, the generated score is diagnostic of liver cancer stages. In certain embodiments, the generated score is diagnostic of hepatocellular carcinoma (HCC). In certain embodiments, the generated score is differentially diagnostic of liver disease comprising cirrhosis. In certain embodiments, the treatment comprises: surgery, adjuvant chemotherapy, neoadjuvant chemotherapy, radiation therapy, hormone therapy, cytotoxic therapy, immunotherapy, adoptive T cell therapy, targeted therapy, and combinations thereof.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The terms “aligned”, “alignment”, “mapped” or “aligning”, “mapping” refer to one or more sequences that are identified as a match in terms of the order of their nucleic acid molecules to a known sequence from a reference genome. Such alignment can be done manually or by a computer algorithm, examples including the Efficient Local Alignment of Nucleotide Data (ELAND) computer program distributed as part of the Illumina Genomics Analysts pipeline. The matching of a sequence read in aligning can be a 100% sequence match or less than 100% (non-perfect match).

The term “cancer” as used herein is meant, a disease, condition, trait, genotype or phenotype characterized by unregulated cell growth or replication as is known in the art. The terms “neoplasm” and “tumor” refer to an abnormal tissue that grows by cellular proliferation more rapidly than normal and continues to grow after the stimuli that initiated proliferation is removed. Such abnormal tissue shows partial or complete lack of structural organization and functional coordination with the normal tissue which may be either benign (such as a benign tumor) or malignant (such as a malignant tumor). Examples of cancer include liver cancer (including hepatocellular carcinoma (HCC)), lung cancer (including non-small cell lung carcinoma), gastric cancer, colorectal cancer, as well as, for example, leukemias, e.g., acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, bladder cancer, prostate cancer, pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis.

The term “cell free nucleic acid,” “cell free polynucleotide”, “cell free DNA,” or “cfDNA” refers to nucleic acid fragments that circulate in an individual's body (e.g., bloodstream) and originate from one or more healthy cells and/or from one or more cancer cells. Additionally, cfDNA may come from other sources such as viruses, fetuses, etc.

The term “circulating tumor DNA” or “ctDNA” refers to nucleic acid fragments that originate from tumor cells or other types of cancer cells, which may be released into an individual's bloodstream as result of biological processes such as apoptosis or necrosis of dying cells or actively released by viable tumor cells.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

An “effective amount” as used herein, means an amount which provides a therapeutic or prophylactic benefit.

As used herein, the terms “fragmentation profile,” “position dependent differences in fragmentation patterns,” and “differences in fragment size and coverage in a position dependent manner across the genome” are equivalent and can be used interchangeably. In some embodiments, determining a cfDNA fragmentation profile in a mammal can be used for identifying a mammal as having cancer. For example, cfDNA fragments obtained from a mammal (e.g., from a sample obtained from a mammal) can be subjected to low coverage whole-genome sequencing, and the sequenced fragments can be mapped to the genome (e.g., in non-overlapping windows) and assessed to determine a cfDNA fragmentation profile. As described herein, a cfDNA fragmentation profile of a mammal having cancer is more heterogeneous (e.g., in fragment lengths) than a cfDNA fragmentation profile of a healthy mammal (e.g., a mammal not having cancer). As such, this disclosure also provides methods and materials for assessing, monitoring, and/or treating mammals (e.g., humans) having, or suspected of having, cancer. In some embodiments, this document provides methods and materials for identifying a mammal as having cancer. For example, a sample (e.g., a blood sample) obtained from a mammal can be assessed to determine the presence and, optionally, the tissue of origin of the cancer in the mammal based, at least in part, on the cfDNA fragmentation profile of the mammal. In some embodiments, methods and materials for monitoring a mammal as having cancer are provided. For example, a sample (e.g., a blood sample) obtained from a mammal can be assessed to determine the presence of the cancer in the mammal based, at least in part, on the cfDNA fragmentation profile of the mammal. In some embodiments, methods and materials for identifying a mammal as having cancer and administering one or more cancer treatments to the mammal to treat the mammal are provided. For example, a sample (e.g., a blood sample) obtained from a mammal can be assessed to determine if the mammal has cancer based, at least in part, on the cfDNA fragmentation profile of the mammal, and one or more cancer treatments can be administered to the mammal.

As used herein, the “frequency” of mutations is defined as the number of variants per million evaluated positions across all the DNA molecules sequenced.

The term “genomic nucleic acid,” or “genomic DNA,” refers to nucleic acid including chromosomal DNA that originates from one or more healthy (e.g., non-tumor) cells. In various embodiments, genomic DNA can be extracted from a cell derived from a blood cell lineage, such as a white blood cell (WBC).

As used herein, the term “mutational profile” refers to the mutation type and frequency as observed in bins across the genome. Comparison of mutation profiles between genomic regions more commonly altered in cancer and mutation profiles from regions more frequently mutated in normal cfDNA can be used to determine multiregional differences.

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some embodiments, the methods of the invention find use in experimental animals, in veterinary application, and in the development of animal models for disease, including, but not limited to, rodents including mice, rats, and hamsters, and primates.

The term “reference genome” as used herein may refer to a digital or previously identified nucleic acid sequence database, assembled as a representative example of a species or subject. Reference genomes may be assembled from the nucleic acid sequences from multiple subjects, sample or organisms and does not necessarily represent the nucleic acid makeup of a single person. Reference genomes may be used to for mapping of sequencing reads from a sample to chromosomal positions. For example, a reference genome used for human subjects as well as many other organisms is found at the National Center for Biotechnology Information at ncbi.nlm.nih.gov.

The term “read segment” or “read” refers to any nucleotide sequences including sequence reads obtained from an individual and/or nucleotide sequences derived from the initial sequence read from a sample obtained from an individual.

The terms “sample,” “patient sample,” “biological sample,” and the like, encompass a variety of sample types obtained from a patient, individual, or subject and can be used in a diagnostic, prognostic and/or monitoring assay. The patient sample may be obtained from a healthy subject, a diseased patient, or a patient with lung cancer. In certain embodiments, a sample that is “provided” can be obtained by the person (or machine) conducting the assay, or it can have been obtained by another, and transferred to the person (or machine) carrying out the assay. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cord blood, amniotic fluid, cerebrospinal fluid, urine, saliva, stool and synovial fluid), solid tissue samples such as a biopsy specimen or tissue cultures or cells derived therefrom and the progeny thereof. In certain embodiment, a sample comprises cerebrospinal fluid. In a specific embodiment, a sample comprises a blood sample. In another embodiment, a sample comprises a plasma sample. In yet another embodiment, a serum sample is used. The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

The term “sequence reads” refers to nucleotide sequences read from a sample obtained from an individual. Sequence reads can be obtained through various methods known in the art.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week, including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 (incudes FIGS. 1A-1C) are plots demonstrating genome-wide fragmentation profiles reflect underlying chromatin structure. FIG. 1A: Fragmentation profiles of 501 individuals in 473 non-overlapping 5mb genomic regions. Fragmentation profiles for cancer individuals show marked heterogeneity as compared to non-cancer individuals with and without liver disease. FIG. 1B: Comparison of plasma fragmentation features to reference A/B compartments. Track 1 shows A/B compartments extracted from liver cancer tissue (29). Track 2 shows a median liver cancer component extracted from the HCC plasma samples of ten liver patients with high tumor fraction by ichor CAN (57). Track 3 shows the median fragmentation profile in the plasma for these 10 HCC samples and Track 4 shows the median profile for 10 healthy plasma samples. Track 5 shows A/B compartments for lymphoblast cells (29). These five tracks show chromosome 22 as an example, with darker shading indicating informative regions of the genome where the two reference tracks differ in domain (open/closed) or magnitude. FIG. 1C: shows further results of comparison of plasma fragmentation features to reference A/B compartments.

FIG. 2. (Incudes FIGS. 2A-2E) are a series of plots showing the fragmentation profiles in HCC patients highlight liver-specific transcription factors. FIG. 2A: The coverage at and around the transcription factor binding sites (TFBS) for the 9 TFs for which the relative coverage at the binding site that had the highest separation of HCC from non-cancer samples. The mean is plotted for each group, with +/−one standard deviation (SD) shown by shading. These confidence intervals (CI) show separation, highlighting that differences in coverage at a TFBS can provide information on cancer status. FIG. 2B: The coverage at and around the TFBS for the 9 TFs that had the lowest separation of HCC from non-cancer samples in the US/EU cohort. These CI are largely overlapping, reflecting their status as TFBS with poor discrimination. Gene-set enrichment analysis of TFs analyzed in both hepatocellular carcinoma and lung adenocarcinoma showed TFs are selectively enriched in numerous pathways related to liver and lung cancer respectively (FIG. 2C) including Adult Liver Carcinoma and Adenocarcinoma of the Lung (FIGS. 2D, FIG. 2E).

FIG. 3 (includes FIGS. 3A-3C) shows high dimensional fragmentation features reflect liver cancer biology and are incorporated in DELFI machine learning approaches. FIG. 3A: A heatmap reflecting the complexity of genome-wide fragmentation and transcription factor binding site features utilized in the DELFI machine learning approach. Each row represents a sample, while columns show individual genomic features. FIG. 3B: Analysis of copy number changes in tissue from 372 TCGA liver cancers and plasma from 501 individuals reflects biological consistency. Copy number changes that occur in TCGA (red=gains, blue=losses) were also found at the chromosomal arm level in HCC plasma, but not in individuals without cancer. FIG. 3C: Heatmap depicting the contributions of individual genomic regions to the final trained DELFI model. The fragmentation features were summarized as three principal components in the model, while aneuploidy was summarized as arm-level z-scores. The top, middle and right panels depict the coefficients of fragmentation components, arm-level z-scores and transcription factor binding sites respectively in the model. CNV, copy number variation.

FIG. 4 (includes FIGS. 4A-4D) are a series of plots demonstrating that the DELFI machine learning models detect liver cancer with high sensitivity and specificity. FIG. 4A: DELFI scores for the US/EU cohort across liver disease and cancer stage for the screening and surveillance models. Cirrhotic patients have DELFI scores higher than individuals without cancer or with viral hepatitis on average, but lower than all stages of liver cancer. Patients with liver cancer across all stages have relatively high DELFI scores, with stage C individuals uniformly having the highest DELFI scores. FIG. 4B: ROC analyses of the US/EU general population cohort and the high-risk surveillance cohort. FIG. 4C: ROC analyses of the US/EU general population and surveillance cohorts separated by BCLC stage, showing high sensitivity and specificity across stages. FIG. 4D: ROC analyses for the fixed surveillance model applied to the Hong Kong cohort, which includes 90 HCC individuals with HCC (85 with BCLC stage A cancer, and 5 with BCLC stage B cancer), 101 individuals with cirrhosis and viral hepatitis and 32 individuals without cancer or liver disease.

FIG. 5 is a heatmap depicting the contributions of individual genomic regions to the final trained DELFI surveillance model. The fragmentation features were summarized as three principal components in the model, while aneuploidy was summarized as arm-level z-scores. The top panel and right panels depict the variable importance of fragmentation components and arm-level z-scores respectively in the model.

FIGS. 6A and 6B are plots showing that DELFI scores in individuals without cancer are unaffected by age and sex. DELFI scores in individuals (n=293, 45 female, 88 male) without cancer or liver disease by age (FIG. 6A) or sex (FIG. 6B).

FIG. 7 is a series of plots demonstrating that DELFI scores in individuals without cancer are not different across racial or ethnic groups.

FIG. 8 is a plot demonstrating that DELFI scores in cirrhotic patients (n=40) with available information were not related to Child-Pugh scores of cirrhosis severity.

FIG. 9 is a series of plots demonstrating that DELFI scores in individuals without cancer (n=53 in Viral Hepatitis patients, and n=78 in cirrhotic patients) vary with BMI.

FIGS. 10A and 10B show performance of alternative DELFI models with and without the addition of TF binding site relative coverage features. ROC analyses of the surveillance model incorporating transcription factor binding site features from liver-derived CHIP-Seq analyses in the high risk US/EU cohort, and for the screening model without transcription factor binding sites for the general population US/EU cohort (FIG. 10A), and by BCLC stage (FIG. 10B).

FIG. 11 shows a plot demonstrating correlation between rank ordered DELFI scores from the Surveillance and Screening models. There is a high correlation among the rank ordered scores using our high risk and screening DELFI models among all HCC patients (n=75).

FIGS. 12A and 12B are a series of plots demonstrating that DELFI scores in patients with HCC were related to tumor lesion size and number. DELFI scores for HCC patients (n=75) were positively correlated with increasing lesion diameter (FIG. 12A) and showed a positive trend with increasing lesion number (FIG. 12B).

FIG. 13 is a graph demonstrating that the DELFI scores in HCC patients (n=54) with resectable stage disease (BCLC 0, A, and B) did not show differences by etiology of underlying liver disease. Stage C patients (n=21) were not included in this analysis as these patients had an etiology largely related to viral hepatitis (n=14).

FIG. 14 is a plot demonstrating that the DELFI score in patients with HCC is correlated with AFP levels. Overall, 39 of 75 individuals with HCC had AFP levels above the recommended screening threshold of 20 ng/ml, indicated by the vertical dotted line. All but three of these individuals would have been detected at the DELFI threshold corresponding to an 80% specificity in the high-risk cohort (0.26), indicated by the horizontal dotted line. Among individuals that would have been undetected by AFP, DELFI detected 30 of 36.

FIG. 15 is a plot demonstrating the correlation of fragmentation profiles to median non-cancer profiles does not differ amongst non-cancer subgroups.

FIG. 16 is a plot demonstrating that the genome wide fragmentation profiles in the Hong Kong validation cohort show similarities to those of the US/EU cohort. Fragmentation profiles of 223 individuals in 473 non-overlapping 5mb genomic regions. Fragmentation profiles for cancer individuals show marked heterogeneity as compared to non-cancer individuals with and without liver disease, similar to observations in the US/EU cohort.

FIG. 17 shows the chromosomal copy number changes detected in plasma are biologically consistent across the US/EU and Hong Kong cohorts as well as in TCGA tissue samples. Red indicates copy number gains, while blue indicates copy number losses. These changes are readily observed in both tissue and HCC plasma, but not evident in individuals without cancer. CNV, copy number variation.

FIG. 18 (includes FIGS. 18A-18D) shows modeling the implementation of DELFI in liver cancer screening. FIG. 18A: The uncertainty of sensitivity and specificity of ultrasound and AFP as well as DELFI screening was modeled in a theoretical population of 100,000 high-risk individuals. Predictive distributions for the number of liver cancers detected (FIG. 18B), false negative rate (FIG. 18C), and negative predictive values (FIG. 18D) among these individuals incorporated variation in both the prevalence of liver cancer and adherence to image- and blood-based screening. The center line in the boxplots represents the median, the upper limit of the boxplots represents the third quantile (75th percentile), the lower limit of the boxplots represents the first quantile (25th percentile), the upper whiskers is the maximum value of the data that is within 1.5 times the interquartile range over the 75th percentile, and the lower whisker is the minimum value of the data that is within 1.5 times the interquartile range under the 25th percentile. shows the chromosomal copy number changes detected in plasma are biologically consistent across the US/EU and Hong Kong cohorts as well as in TCGA tissue samples. Red indicates copy number gains, while blue indicates copy number losses. These changes are readily observed in both tissue and HCC plasma, but not evident in individuals without cancer. CNV, copy number variation.

### DETAILED DESCRIPTION

DELFI (DNA evaluation of fragments for early interception) utilizes genome-wide fragmentation profiles to provide a high performing and cost-effective approach for cancer detection (21,22). Fragmentation and methylation information has also demonstrated the ability to differentiate patients with liver cancer from those without cancer (24), although such an approach requires two distinct methods of cfDNA library preparation and analysis. To date, no study has validated genome-wide approaches for detecting HCC in independent groups or across different high-risk populations.

Accordingly, in certain embodiments, a method of diagnosing liver diseases or disorders in a subject, comprises isolating circulating cell free DNA (cfDNA) from a subject; conducting whole genome sequencing of cfDNA molecules to generate genomic libraries and fragmentation profiles; comparing the fragmentation profiles to healthy subjects and/or reference genome, and diagnosing whether the subject has a liver disease or disorder.

In another embodiment, a method of diagnosing liver cancer, comprises isolating circulating cell free DNA (cfDNA) from a biological sample and conducting whole genome sequencing of cfDNA molecules to generate genomic libraries and fragmentation profiles; identifying cellular origins of the cfDNA fragmentation profiles comprising comparing genome-wide fragmentome profiles to high-throughput sequencing chromosome conformation capture (Hi-C); correlating cfDNA fragmentation profiles with altered DNA binding of transcription factors; determining chromosomal gains or losses in liver cancer subjects as compared to healthy subjects; executing a machine learning model for determining changes in cfDNA fragmentation profiles that classifies the subject as a cancer patient based on the cfDNA fragmentation profile for the subject; thereby, diagnosing liver cancer and administering a cancer treatment to the subject.

cfDNA Fragmentation Profiles

A cfDNA fragmentation profile can include one or more cfDNA fragmentation patterns. A cfDNA fragmentation pattern can include any appropriate cfDNA fragmentation pattern. Examples of cfDNA fragmentation patterns include, without limitation, median fragment size, fragment size distribution, ratio of small cfDNA fragments to large cfDNA fragments, and the coverage of cfDNA fragments. In some embodiments, a cfDNA fragmentation pattern includes two or more (e.g., two, three, or four) of median fragment size, fragment size distribution, ratio of small cfDNA fragments to large cfDNA fragments, and the coverage of cfDNA fragments. In some embodiments, cfDNA fragmentation profile can be a genome-wide cfDNA profile (e.g., a genome-wide cfDNA profile in windows across the genome). In some embodiments, cfDNA fragmentation profile can be a targeted region profile. A targeted region can be any appropriate portion of the genome (e.g., a chromosomal region). Examples of chromosomal regions for which a cfDNA fragmentation profile can be determined as described herein include, without limitation, a portion of a chromosome (e.g., a portion of 2q, 4p, 5p, 6q, 7p, 8q, 9q, 10q, 11q, 12q, and/or 14q) and a chromosomal arm (e.g., a chromosomal arm of 8q, 13q, 11q, and/or 3p). In some embodiments, a cfDNA fragmentation profile can include two or more targeted region profiles.

In some embodiments, a cfDNA fragmentation profile can be used to identify changes (e.g., alterations) in cfDNA fragment lengths. An alteration can be a genome-wide alteration or an alteration in one or more targeted regions/loci. A target region can be any region containing one or more cancer-specific alterations. In some embodiments, a cfDNA fragmentation profile can be used to identify (e.g., simultaneously identify) from about 10 alterations to about 500 alterations (e.g., from about 25 to about 500, from about 50 to about 500, from about 100 to about 500, from about 200 to about 500, from about 300 to about 500, from about 10 to about 400, from about 10 to about 300, from about 10 to about 200, from about 10 to about 100, from about 10 to about 50, from about 20 to about 400, from about 30 to about 300, from about 40 to about 200, from about 50 to about 100, from about 20 to about 100, from about 25 to about 75, from about 50 to about 250, or from about 100 to about 200, alterations).

A cfDNA fragmentation profile can be obtained using any appropriate method. In some embodiments, cfDNA from a mammal (e.g., a mammal having, or suspected of having, cancer) can be processed into sequencing libraries which can be subjected to whole genome sequencing (e.g., low-coverage whole genome sequencing), mapped to the genome, and analyzed to determine cfDNA fragment lengths. Mapped sequences can be analyzed in non-overlapping windows covering the genome. Windows can be any appropriate size. For example, windows can be from thousands to millions of bases in length. As one non-limiting example, a window can be about 5 megabases (Mb) long. Any appropriate number of windows can be mapped. For example, tens to thousands of windows can be mapped in the genome. For example, hundreds to thousands of windows can be mapped in the genome. A cfDNA fragmentation profile can be determined within each window.

In some embodiments, methods and materials described herein also can include machine learning. For example, machine learning can be used for identifying mutation frequencies, altered fragmentation profile (e.g., using coverage of cfDNA fragments, fragment size of cfDNA fragments, coverage of chromosomes, and mtDNA).

In some embodiments, determining a cfDNA fragmentation profile in a mammal can be used for identifying a mammal as having cancer. For example, cfDNA fragments obtained from a mammal (e.g., from a sample obtained from a mammal) can be subjected to low coverage whole-genome sequencing, and the sequenced fragments can be mapped to the genome and assessed to determine a cfDNA fragmentation profile. As described herein, a cfDNA fragmentation profile of a mammal having cancer is more heterogeneous (e.g., in fragment lengths) than a cfDNA fragmentation profile of a healthy mammal (e.g., a mammal not having cancer). As such, also provided are methods and materials for assessing, monitoring, and/or treating mammals (e.g., humans) having, or suspected of having, cancer. In some embodiments, methods and materials are provided for identifying a mammal as having cancer. For example, a sample (e.g., a blood sample) obtained from a mammal can be assessed to determine the presence and, optionally, the tissue of origin of the cancer in the mammal based, at least in part, on the cfDNA fragmentation profile of the mammal. In some embodiments, methods and materials are provided for monitoring a mammal as having cancer. For example, a sample (e.g., a blood sample) obtained from a mammal can be assessed to determine the presence of the cancer in the mammal based, at least in part, on the cfDNA fragmentation profile of the mammal. In some embodiments, methods and materials are provided for identifying a mammal as having cancer and administering one or more cancer treatments to the mammal to treat the mammal. For example, a sample (e.g., a blood sample) obtained from a mammal can be assessed to determine if the mammal has cancer based, at least in part, on the cfDNA fragmentation profile of the mammal, and one or more cancer treatments can be administered to the mammal.

In some embodiments, a cfDNA fragmentation profile can be used to detect tumor-derived DNA. For example, a cfDNA fragmentation profile can be used to detect tumor-derived DNA by comparing a cfDNA fragmentation profile of a mammal having, or suspected of having, cancer to a reference cfDNA fragmentation profile (e.g., a cfDNA fragmentation profile of a healthy mammal and/or a nucleosomal DNA fragmentation profile of healthy cells from the mammal having, or suspected of having, cancer). In some embodiments, a reference cfDNA fragmentation profile is a previously generated profile from a healthy mammal. For example, methods provided herein can be used to determine a reference cfDNA fragmentation profile in a healthy mammal, and that reference cfDNA fragmentation profile can be stored (e.g., in a computer or other electronic storage medium) for future comparison to a test cfDNA fragmentation profile in mammal having, or suspected of having, cancer. In some embodiments, a reference cfDNA fragmentation profile (e.g., a stored cfDNA fragmentation profile) of a healthy mammal is determined over the whole genome. In some embodiments, a reference cfDNA fragmentation profile (e.g., a stored cfDNA fragmentation profile) of a healthy mammal is determined over a subgenomic interval.

In some embodiments, a cfDNA fragmentation profile can be used to identify a mammal (e.g., a human) as having cancer (e.g., a liver cancer, a colorectal cancer, a lung cancer, a breast cancer, a gastric cancer, a pancreatic cancer, a bile duct cancer, and/or an ovarian cancer).

A cfDNA fragmentation profile can include a cfDNA fragment size pattern. cfDNA fragments can be any appropriate size. For example, cfDNA fragment can be from about 50 base pairs (bp) to about 400 bp in length.

A cfDNA fragmentation profile can include a cfDNA fragment size distribution. As described herein, a mammal having cancer can have a cfDNA size distribution that is more variable than a cfDNA fragment size distribution in a healthy mammal. In some embodiments, a size distribution can be within a targeted region. A healthy mammal (e.g., a mammal not having cancer) can have a targeted region cfDNA fragment size distribution of about 1 or less than about 1. In some embodiments, a mammal having cancer can have a targeted region cfDNA fragment size distribution that is longer (e.g., 10, 15, 20, 25, 30, 35, 40, 45, 50 or more bp longer, or any number of base pairs between these numbers) than a targeted region cfDNA fragment size distribution in a healthy mammal. In some embodiments, a mammal having cancer can have a targeted region cfDNA fragment size distribution that is shorter (e.g., 10, 15, 20, 25, 30, 35, 40, 45, 50 or more bp shorter, or any number of base pairs between these numbers) than a targeted region cfDNA fragment size distribution in a healthy mammal. In some embodiments, a size distribution can be a genome-wide size distribution. A healthy mammal (e.g., a mammal not having cancer) can have very similar distributions of short and long cfDNA fragments genome wide. In some embodiments, a mammal having cancer can have, genome-wide, one or more alterations (e.g., increases and decreases) in cfDNA fragment sizes. The one or more alterations can be any appropriate chromosomal region of the genome. For example, an alteration can be in a portion of a chromosome. Examples of portions of chromosomes that can contain one or more alterations in cfDNA fragment sizes include, without limitation, portions of 2q, 4p, 5p, 6q, 7p, 8q, 99, 10q, 11q, 12q, and 14q. For example, an alteration can be across a chromosome arm (e.g., an entire chromosome arm).

A cfDNA fragmentation profile can include a ratio of small cfDNA fragments to large cfDNA fragments and a correlation of fragment ratios to reference fragment ratios. As used herein, with respect to ratios of small cfDNA fragments to large cfDNA fragments, a small cfDNA fragment can be from about 100 bp in length to about 150 bp in length. As used herein, with respect to ratios of small cfDNA fragments to large cfDNA fragments, a large cfDNA fragment can be from about 151 bp in length to 220 bp in length. A mammal having cancer can have a correlation of fragment ratios (e.g., a correlation of cfDNA fragment ratios to reference DNA fragment ratios such as DNA fragment ratios from one or more healthy mammals) that is lower (e.g., 2-fold lower, 3-fold lower, 4-fold lower, 5-fold lower, 6-fold lower, 7-fold lower, 8-fold lower, 9-fold lower, 10-fold lower, or more) than in a healthy mammal. A healthy mammal (e.g., a mammal not having cancer) can have a correlation of fragment ratios (e.g., a correlation of cfDNA fragment ratios to reference DNA fragment ratios such as DNA fragment ratios from one or more healthy mammals) of about 1 (e.g., about 0.96). In some embodiments, a mammal having cancer can have a correlation of fragment ratios (e.g., a correlation of cfDNA fragment ratios to reference DNA fragment ratios such as DNA fragment ratios from one or more healthy mammals) that is, on average, lower than a correlation of fragment ratios (e.g., a correlation of cfDNA fragment ratios to reference DNA fragment ratios such as DNA fragment ratios from one or more healthy mammals) in a healthy mammal.

A cfDNA fragmentation profile can include coverage of all fragments. Coverage of all fragments can include windows (e.g., non-overlapping windows) of coverage. In some embodiments, coverage of all fragments can include windows of small fragments (e.g., fragments from about 100 bp to about 150 bp in length). In some embodiments, coverage of all fragments can include windows of large fragments (e.g., fragments from about 151 bp to about 220 bp in length).

In certain embodiments, a cfDNA fragmentation profile can be used to identify the molecular origins of cfDNA in patients and identify genomic and chromatin features associated with fragmentation changes.

In some embodiments, a cfDNA fragmentation profile can be used to identify the tissue of origin of a cancer (e.g., a liver cancer, a colorectal cancer, a lung cancer, a breast cancer, a gastric cancer, a pancreatic cancer, a bile duct cancer, or an ovarian cancer). For example, a cfDNA fragmentation profile can be used to identify a localized cancer. When a cfDNA fragmentation profile includes a targeted region profile, one or more alterations described herein can be used to identify the tissue of origin of a cancer. In some embodiments, one or more alterations in chromosomal regions can be used to identify the tissue of origin of a cancer.

A cfDNA fragmentation profile can be obtained using any appropriate method. In some embodiments, cfDNA from a mammal (e.g., a mammal having, or suspected of having, cancer) can be processed into sequencing libraries which can be subjected to whole genome sequencing (e.g., low-coverage whole genome sequencing), mapped to the genome, and analyzed to determine cfDNA fragment lengths. Mapped sequences can be analyzed in non-overlapping windows covering the genome. Windows can be any appropriate size. For example, windows can be from thousands to millions of bases in length. As one non-limiting example, a window can be about 5 megabases (Mb) long. Any appropriate number of windows can be mapped. For example, tens to thousands of windows can be mapped in the genome. For example, hundreds to thousands of windows can be mapped in the genome. A cfDNA fragmentation profile can be determined within each window.

In some embodiments, methods and materials described herein also can include machine learning. For example, machine learning can be used for identifying an altered fragmentation profile (e.g., using coverage of cfDNA fragments, fragment size of cfDNA fragments, coverage of chromosomes, and mtDNA).

In some embodiments, methods and materials described herein can be the sole method used to identify a mammal (e.g., a human) as having liver cancer. For example, determining a cfDNA fragmentation profile can be the sole method used to identify a mammal as having liver cancer.

In some embodiments, methods and materials described herein can be used together with one or more additional methods used to identify a mammal (e.g., a human) as having liver cancer. Examples of methods used to identify a mammal as having cancer include, without limitation, identifying one or more cancer-specific sequence alterations, identifying one or more chromosomal alterations (e.g., aneuploidies and rearrangements), and identifying other cfDNA alterations. For example, determining a cfDNA fragmentation profile can be used together with identifying one or more cancer-specific mutations in a mammal's genome to identify a mammal as having liver cancer. For example, determining a cfDNA fragmentation profile can be used together with identifying one or more aneuploidies in a mammal's genome to identify a mammal as having liver cancer.

### Methods of Treatment

The methods embodied herein, include identifying a mammal as having cancer. The methods include, extracting cell-free DNA (cfDNA) from a subject's biological sample; generating genomic libraries from the extracted cfDNA; sequencing individual cfDNA molecules to obtain fragmentation profiles; comparing the fragmentation profiles to healthy subjects and/or reference genome, and diagnosing whether the subject has a liver disease or disorder.

In certain embodiments, a method of diagnosing liver cancer, comprises isolating circulating cell free DNA (cfDNA) from a biological sample and conducting whole genome sequencing of cfDNA molecules to generate genomic libraries and fragmentation profiles; identifying cellular origins of the cfDNA fragmentation profiles comprising comparing genome-wide fragmentome profiles to high-throughput sequencing chromosome conformation capture (Hi-C); correlating cfDNA fragmentation profiles with altered DNA binding of transcription factors; determining chromosomal gains or losses in liver cancer subjects as compared to healthy subjects; executing a machine learning model for determining changes in cfDNA fragmentation profiles that classifies the subject as a cancer patient based on the cfDNA fragmentation profile for the subject; thereby, diagnosing liver cancer and administering a cancer treatment to the subject.

In certain embodiments, a subject is diagnosed as having cancer, e.g. early stage cancer. In certain embodiments, the type and stage of liver cancer is identified and the subject is treated with one or more cancer therapies.

In some embodiments, methods and materials described herein for assessing, monitoring, and/or treating mammals (e.g., humans) having, or suspected of having, cancer. In some embodiments, methods and materials are provided for identifying a mammal as having liver cancer. For example, a sample (e.g., a blood sample) obtained from a mammal can be assessed to determine if the mammal has cancer based, at least in part, on the cfDNA fragmentation profile of the mammal. In some embodiments, a sample (e.g., a blood sample) obtained from a mammal can be assessed to determine the tissue of origin of the cancer in the mammal based, at least in part, on the cfDNA fragmentation profile of the mammal. In some embodiments, methods and materials are provided for identifying a mammal as having liver cancer and administering one or more treatments to the mammal to treat the mammal. For example, a sample (e.g., a blood sample) obtained from a mammal can be assessed to determine if the mammal has liver cancer based, at least in part, on the cfDNA fragmentation profile of the mammal, and administering one or more cancer treatments to the mammal. In some embodiments, methods and materials are provided for treating a mammal having cancer. For example, one or more cancer treatments can be administered to a mammal identified as having cancer (e.g., based, at least in part, on the cfDNA fragmentation profile of the mammal) to treat the mammal. In some embodiments, during or after the course of a cancer treatment (e.g., any of the cancer treatments described herein), a mammal can undergo monitoring (or be selected for increased monitoring) and/or further diagnostic testing. In some embodiments, monitoring can include assessing mammals having, or suspected of having, liver cancer by, for example, assessing a sample (e.g., a blood sample) obtained from the mammal to determine the cfDNA fragmentation profile of the mammal as described herein, and changes in the cfDNA fragmentation profiles over time can be used to identify response to treatment and/or identify the mammal as having cancer (e.g., a residual cancer).

Any appropriate mammal can be assessed, monitored, and/or treated as described herein. A mammal can be a mammal having liver cancer. A mammal can be a mammal suspected of having liver cancer. Examples of mammals that can be assessed, monitored, and/or treated as described herein include, without limitation, humans, primates such as monkeys, dogs, cats, horses, cows, pigs, sheep, mice, and rats. For example, a human having, or suspected of having, liver cancer can be assessed to determine a cfDNA fragmentation profiled as described herein and, optionally, can be treated with one or more cancer treatments as described herein.

Any appropriate sample from a mammal can be assessed as described herein (e.g., assessed for a DNA fragmentation pattern). In some embodiments, a sample can include DNA (e.g., genomic DNA). In some embodiments, a sample can include cfDNA (e.g., circulating tumor DNA (ctDNA)). In some embodiments, a sample can be fluid sample (e.g., a liquid biopsy). Examples of samples that can contain DNA and/or polypeptides include, without limitation, blood (e.g., whole blood, serum, or plasma), amnion, tissue, urine, cerebrospinal fluid, saliva, sputum, broncho-alveolar lavage, bile, lymphatic fluid, cyst fluid, stool, ascites, pap smears, breast milk, and exhaled breath condensate. For example, a plasma sample can be assessed to determine a cfDNA fragmentation profiled as described herein.

A sample from a mammal to be assessed as described herein (e.g., assessed for a DNA fragmentation pattern) can include any appropriate amount of cfDNA. In some embodiments, a sample can include a limited amount of DNA. For example, a cfDNA fragmentation profile can be obtained from a sample that includes less DNA than is typically required for other cfDNA analysis methods, such as those described in, for example, Phallen et al., 2017 Sci Transl Med 9; Cohen et al., 2018 Science 359:926; Newman et al., 2014 Nat Med 20:548; and Newman et al., 2016 Nat Biotechnol 34:547).

In some embodiments, a sample can be processed (e.g., to isolate and/or purify DNA and/or polypeptides from the sample). For example, DNA isolation and/or purification can include cell lysis (e.g., using detergents and/or surfactants), protein removal (e.g., using a protease), and/or RNA removal (e.g., using an RNase). As another example, polypeptide isolation and/or purification can include cell lysis (e.g., using detergents and/or surfactants), DNA removal (e.g., using a DNase), and/or RNA removal (e.g., using an RNase).

A cancer can be any stage cancer. In some embodiments, a cancer can be an early-stage cancer. In some embodiments, a cancer can be an asymptomatic cancer. In some embodiments, a cancer can be a residual disease and/or a recurrence (e.g., after surgical resection and/or after cancer therapy). A cancer can be any type of cancer. Examples of types of cancers that can be assessed, monitored, and/or treated as described herein include, without limitation, colorectal cancers, lung cancers, breast cancers, gastric cancers, pancreatic cancers, bile duct cancers, and ovarian cancers.

When treating a mammal having, or suspected of having, liver cancer as described herein, the mammal can be administered one or more cancer treatments. A cancer treatment can be any appropriate cancer treatment. One or more cancer treatments described herein can be administered to a mammal at any appropriate frequency (e.g., once or multiple times over a period of time ranging from days to weeks). Examples of cancer treatments include, without limitation adjuvant chemotherapy, neoadjuvant chemotherapy, radiation therapy, hormone therapy, cytotoxic therapy, immunotherapy, adoptive T cell therapy (e.g., chimeric antigen receptors and/or T cells having wild-type or modified T cell receptors), targeted therapy such as administration of kinase inhibitors (e.g., kinase inhibitors that target a particular genetic lesion, such as a translocation or mutation), (e.g. a kinase inhibitor, an antibody, a bispecific antibody), signal transduction inhibitors, bispecific antibodies or antibody fragments (e.g., BiTEs), monoclonal antibodies, immune checkpoint inhibitors, surgery (e.g., surgical resection), or any combination of the above. In some embodiments, a cancer treatment can reduce the severity of the cancer, reduce a symptom of the cancer, and/or to reduce the number of cancer cells present within the mammal.

In some embodiments, a cancer treatment can include an immune checkpoint inhibitor. Non-limiting examples of immune checkpoint inhibitors include nivolumab (Opdivo), pembrolizumab (Keytruda), atezolizumab (tecentriq), avelumab (bavencio), durvalumab (imfinzi), ipilimumab (yervoy).

Cancer therapies in general also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The chemotherapy may also be administered at low, continuous doses which is known as metronomic chemotherapy.

Yet further combination chemotherapies include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; carboplatin, procarbazine, plicomycin, gemcitabien, navelbine, farnesyl-protein transferase inhibitors, transplatinum; and pharmaceutically acceptable salts, acids or derivatives of any of the above.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells as well as genetically engineered variants of these cell types modified to express chimeric antigen receptors.

The immunotherapy may comprise suppression of T regulatory cells (Tregs), myeloid derived suppressor cells (MDSCs) and cancer associated fibroblasts (CAFs). In some embodiments, the immunotherapy is a tumor vaccine (e.g., whole tumor cell vaccines, peptides, and recombinant tumor associated antigen vaccines), or adoptive cellular therapies (ACT) (e.g., T cells, natural killer cells, TILs, and LAK cells). The T cells may be engineered with chimeric antigen receptors (CARs) or T cell receptors (TCRs) to specific tumor antigens. As used herein, a chimeric antigen receptor (or CAR) may refer to any engineered receptor specific for an antigen of interest that, when expressed in a T cell, confers the specificity of the CAR onto the T cell. Once created using standard molecular techniques, a T cell expressing a chimeric antigen receptor may be introduced into a patient, as with a technique such as adoptive cell transfer. In some aspects, the T cells are activated CD4 and/or CD8 T cells in the individual which are characterized by γ-IFN-producing CD4 and/or CD8 T cells and/or enhanced cytolytic activity relative to prior to the administration of the combination. The CD4 and/or CD8 T cells may exhibit increased release of cytokines selected from the group consisting of IFN-γ, TNF-α and interleukins. The CD4 and/or CD8 T cells can be effector memory T cells. In certain embodiments, the CD4 and/or CD8 effector memory T cells are characterized by having the expression of CD44^{high} CD62L^{low}.

The immunotherapy may be a cancer vaccine comprising one or more cancer antigens, in particular a protein or an immunogenic fragment thereof, DNA or RNA encoding said cancer antigen, in particular a protein or an immunogenic fragment thereof, cancer cell lysates, and/or protein preparations from tumor cells. As used herein, a cancer antigen is an antigenic substance present in cancer cells. In principle, any protein produced in a cancer cell that has an abnormal structure due to mutation can act as a cancer antigen. In principle, cancer antigens can be products of mutated Oncogenes and tumor suppressor genes, products of other mutated genes, overexpressed or aberrantly expressed cellular proteins, cancer antigens produced by oncogenic viruses, oncofetal antigens, altered cell surface glycolipids and glycoproteins, or cell type-specific differentiation antigens. Examples of cancer antigens include the abnormal products of ras and p53 genes. Other examples include tissue differentiation antigens, mutant protein antigens, oncogenic viral antigens, cancer-testis antigens and vascular or stromal specific antigens. Tissue differentiation antigens are those that are specific to a certain type of tissue.

The immunotherapy may be an antibody, such as part of a polyclonal antibody preparation, or may be a monoclonal antibody. The antibody may be a humanized antibody, a chimeric antibody, an antibody fragment, a bispecific antibody or a single chain antibody. An antibody as disclosed herein includes an antibody fragment, such as, but not limited to, Fab, Fab′ and F(ab′)2, Fd, single-chain Fvs (scFv), single-chain antibodies, disulfide-linked Fvs (sdfv) and fragments including either a VL or VH domain. In some aspects, the antibody or fragment thereof specifically binds epidermal growth factor receptor (EGFR1, Erb-B1), HER2/neu (Erb-B2), CD20, Vascular endothelial growth factor (VEGF), insulin-like growth factor receptor (IGF-1R), TRAIL-receptor, epithelial cell adhesion molecule, carcino-embryonic antigen, Prostate-specific membrane antigen, Mucin-1, CD30, CD33, or CD40.

Examples of monoclonal antibodies include, without limitation, trastuzumab (anti-HER2/neu antibody); Pertuzumab (anti-HER2 mAb); cetuximab (chimeric monoclonal antibody to epidermal growth factor receptor EGFR); panitumumab (anti-EGFR antibody); nimotuzumab (anti-EGFR antibody); Zalutumumab (anti-EGFR mAb); Necitumumab (anti-EGFR mAb); MDX-210 (humanized anti-HER-2 bispecific antibody); MDX-210 (humanized anti-HER-2 bispecific antibody); MDX-447 (humanized anti-EGF receptor bispecific antibody); Rituximab (chimeric murine/human anti-CD20 mAb); Obinutuzumab (anti-CD20 mAb); Ofatumumab (anti-CD20 mAb); Tositumumab-I131 (anti-CD20 mAb); Ibritumomab tiuxetan (anti-CD20 mAb); Bevacizumab (anti-VEGF mAb); Ramucirumab (anti-VEGFR2 mAb); Ranibizumab (anti-VEGF mAb); Aflibercept (extracellular domains of VEGFR1 and VEGFR2 fused to IgG1 Fc); AMG386 (angiopoietin-1 and -2 binding peptide fused to IgG1 Fc); Dalotuzumab (anti-IGF-1R mAb); Gemtuzumab ozogamicin (anti-CD33 mAb); Alemtuzumab (anti-Campath-1/CD52 mAb); Brentuximab vedotin (anti-CD30 mAb); Catumaxomab (bispecific mAb that targets epithelial cell adhesion molecule and CD3); Naptumomab (anti-5T4 mAb); Girentuximab (anti-Carbonic anhydrase ix); or Farletuzumab (anti-folate receptor). Other examples include antibodies such as Panorex™ (17-1A) (murine monoclonal antibody); Panorex (MAb17-1A) (chimeric murine monoclonal antibody); BEC2 (ami-idiotypic mAb, mimics the GD epitope) (with BCG); Oncolym (Lym-1 monoclonal antibody); SMART M195 Ab, humanized 13′ 1 LYM-1 (Oncolym), Ovarex (B43.13, anti-idiotypic mouse mAb); 3622W94 mAb that binds to EGP40 (17-1A) pancarcinoma antigen on adenocarcinomas; Zenapax (SMART Anti-Tac (IL-2 receptor); SMART M195 Ab, humanized Ab, humanized); NovoMAb-G2 (pancarcinoma specific Ab); TNT (chimeric mAb to histone antigens); TNT (chimeric mAb to histone antigens); Gliomab-H (Monoclonals-Humanized Abs); GNI-250 Mab; EMD-72000 (chimeric-EGF antagonist); LymphoCide (humanized IL.L.2 antibody); and MDX-260 bispecific, targets GD-2, ANA Ab, SMART IDIO Ab, SMART ABL 364 Ab or ImmuRAIT-CEA. Further examples of antibodies include Zanulimumab (anti-CD4 mAb), Keliximab (anti-CD4 mAb); Ipilimumab (MDX-101; anti-CTLA-4 mAb); Tremilimumab (anti-CTLA-4 mAb); (Daclizumab (anti-CD25/IL-2R mAb); Basiliximab (anti-CD25/IL-2R mAb); MDX-1106 (anti-PD1 mAb); antibody to GITR; GC1008 (anti-TGF-β antibody); metelimumab/CAT-192 (anti-TGF-β antibody); lerdelimumab/CAT-152 (anti-TGF-β antibody); ID11 (anti-TGF-β antibody); Denosumab (anti-RANKL mAb); BMS-663513 (humanized anti-4-1BB mAb); SGN-40 (humanized anti-CD40 mAb); CP870,893 (human anti-CD40 mAb); Infliximab (chimeric anti-TNF mAb; Adalimumab (human anti-TNF mAb); Certolizumab (humanized Fab anti-TNF); Golimumab (anti-TNF); Etanercept (Extracellular domain of TNFR fused to IgG1 Fc); Belatacept (Extracellular domain of CTLA-4 fused to Fc); Abatacept (Extracellular domain of CTLA-4 fused to Fc); Belimumab (anti-B Lymphocyte stimulator); Muromonab-CD3 (anti-CD3 mAb); Otelixizumab (anti-CD3 mAb); Teplizumab (anti-CD3 mAb); Tocilizumab (anti-IL6R mAb); REGN88 (anti-IL6R mAb); Ustekinumab (anti-IL-12/23 mAb); Briakinumab (anti-IL-12/23 mAb); Natalizumab (anti-α4 integrin); Vedolizumab (anti-α4 β7 integrin mAb); T1 h (anti-CD6 mAb); Epratuzumab (anti-CD22 mAb); Efalizumab (anti-CD11a mAb); and Atacicept (extracellular domain of transmembrane activator and calcium-modulating ligand interactor fused with Fc).

When monitoring a mammal having, or suspected of having, cancer as described herein (e.g., based, at least in part, on the cfDNA fragmentation profile of the mammal), the monitoring can be before, during, and/or after the course of a cancer treatment. Methods of monitoring provided herein can be used to determine the efficacy of one or more cancer treatments and/or to select a mammal for increased monitoring. In some embodiments, the monitoring can include identifying a cfDNA fragmentation profile as described herein. For example, a cfDNA fragmentation profile can be obtained before administering one or more cancer treatments to a mammal having, or suspected or having, cancer, one or more cancer treatments can be administered to the mammal, and one or more cfDNA fragmentation profiles can be obtained during the course of the cancer treatment. In some embodiments, a cfDNA fragmentation profile can change during the course of cancer treatment (e.g., any of the cancer treatments described herein). For example, a cfDNA fragmentation profile indicative that the mammal has cancer can change to a cfDNA fragmentation profile indicative that the mammal does not have cancer. Such a cfDNA fragmentation profile change can indicate that the cancer treatment is working. Conversely, a cfDNA fragmentation profile can remain static (e.g., the same or approximately the same) during the course of cancer treatment (e.g., any of the cancer treatments described herein). Such a static cfDNA fragmentation profile can indicate that the cancer treatment is not working.

In some embodiments, the monitoring can include conventional techniques capable of monitoring one or more cancer treatments (e.g., the efficacy of one or more cancer treatments). In some embodiments, a mammal selected for increased monitoring can be administered a diagnostic test (e.g., any of the diagnostic tests disclosed herein) at an increased frequency compared to a mammal that has not been selected for increased monitoring. For example, a mammal selected for increased monitoring can be administered a diagnostic test at a frequency of twice daily, daily, bi-weekly, weekly, bi-monthly, monthly, quarterly, semi-annually, annually, or any at frequency therein. In some embodiments, a mammal selected for increased monitoring can be administered a one or more additional diagnostic tests compared to a mammal that has not been selected for increased monitoring. For example, a mammal selected for increased monitoring can be administered two diagnostic tests, whereas a mammal that has not been selected for increased monitoring is administered only a single diagnostic test (or no diagnostic tests). In some embodiments, a mammal that has been selected for increased monitoring can also be selected for further diagnostic testing. Once the presence of a tumor or a cancer (e.g., a cancer cell) has been identified (e.g., by any of the variety of methods disclosed herein), it may be beneficial for the mammal to undergo both increased monitoring (e.g., to assess the progression of the tumor or cancer in the mammal and/or to assess the development of one or more cancer biomarkers such as mutations), and further diagnostic testing (e.g., to determine the size and/or exact location (e.g., tissue of origin) of the tumor or the cancer). In some embodiments, one or more cancer treatments can be administered to the mammal that is selected for increased monitoring after a cancer biomarker is detected and/or after the cfDNA fragmentation profile of the mammal has not improved or deteriorated. Any of the cancer treatments disclosed herein or known in the art can be administered. For example, a mammal that has been selected for increased monitoring can be further monitored, and a cancer treatment can be administered if the presence of the cancer cell is maintained throughout the increased monitoring period. Additionally, or alternatively, a mammal that has been selected for increased monitoring can be administered a cancer treatment, and further monitored as the cancer treatment progresses. In some embodiments, after a mammal that has been selected for increased monitoring has been administered a cancer treatment, the increased monitoring will reveal one or more cancer biomarkers (e.g., mutations). In some embodiments, such one or more cancer biomarkers will provide cause to administer a different cancer treatment (e.g., a resistance mutation may arise in a cancer cell during the cancer treatment, which cancer cell harboring the resistance mutation is resistant to the original cancer treatment).

When a mammal is identified as having cancer as described herein (e.g., based, at least in part, on the cfDNA fragmentation profile of the mammal), the identifying can be before and/or during the course of a cancer treatment. Methods of identifying a mammal as having cancer provided herein can be used as a first diagnosis to identify the mammal (e.g., as having cancer before any course of treatment) and/or to select the mammal for further diagnostic testing. In some embodiments, once a mammal has been determined to have cancer, the mammal may be administered further tests and/or selected for further diagnostic testing. In some embodiments, methods provided herein can be used to select a mammal for further diagnostic testing at a time period prior to the time period when conventional techniques are capable of diagnosing the mammal with an early-stage cancer. For example, methods provided herein for selecting a mammal for further diagnostic testing can be used when a mammal has not been diagnosed with cancer by conventional methods and/or when a mammal is not known to harbor a cancer. In some embodiments, a mammal selected for further diagnostic testing can be administered a diagnostic test (e.g., any of the diagnostic tests disclosed herein) at an increased frequency compared to a mammal that has not been selected for further diagnostic testing. For example, a mammal selected for further diagnostic testing can be administered a diagnostic test at a frequency of twice daily, daily, bi-weekly, weekly, bi-monthly, monthly, quarterly, semi-annually, annually, or any at frequency therein. In some embodiments, a mammal selected for further diagnostic testing can be administered a one or more additional diagnostic tests compared to a mammal that has not been selected for further diagnostic testing. For example, a mammal selected for further diagnostic testing can be administered two diagnostic tests, whereas a mammal that has not been selected for further diagnostic testing is administered only a single diagnostic test (or no diagnostic tests). In some embodiments, the diagnostic testing method can determine the presence of the same type of cancer (e.g., having the same tissue or origin) as the cancer that was originally detected (e.g., based, at least in part, on the cfDNA fragmentation profile of the mammal). Additionally, or alternatively, the diagnostic testing method can determine the presence of a different type of cancer as the cancer that was original detected. In some embodiments, the diagnostic testing method is a scan. In some embodiments, the scan is a computed tomography (CT), a CT angiography (CTA), an esophagram (a Barium swallow), a Barium enema, a magnetic resonance imaging (MRI), a PET scan, an ultrasound (e.g., an endobronchial ultrasound, an endoscopic ultrasound), an X-ray, a DEXA scan.

In some embodiments, the diagnostic testing method is a physical examination, such as an anoscopy, a bronchoscopy (e.g., an autofluorescence bronchoscopy, a white-light bronchoscopy, a navigational bronchoscopy), a colonoscopy, a digital breast tomosynthesis, an endoscopic retrograde cholangiopancreatography (ERCP), an ensophagogastroduodenoscopy, a mammography, a Pap smear, a pelvic exam, a positron emission tomography and computed tomography (PET-CT) scan. In some embodiments, a mammal that has been selected for further diagnostic testing can also be selected for increased monitoring. Once the presence of a tumor or a cancer (e.g., a cancer cell) has been identified (e.g., by any of the variety of methods disclosed herein), it may be beneficial for the mammal to undergo both increased monitoring (e.g., to assess the progression of the tumor or cancer in the mammal and/or to assess the development of one or more cancer biomarkers such as mutations), and further diagnostic testing (e.g., to determine the size and/or exact location of the tumor or the cancer). In some embodiments, a cancer treatment is administered to the mammal that is selected for further diagnostic testing after a cancer biomarker is detected and/or after the cfDNA fragmentation profile of the mammal has not improved or deteriorated. Any of the cancer treatments disclosed herein or known in the art can be administered. For example, a mammal that has been selected for further diagnostic testing can be administered a further diagnostic test, and a cancer treatment can be administered if the presence of the tumor or the cancer is confirmed. Additionally, or alternatively, a mammal that has been selected for further diagnostic testing can be administered a cancer treatment, and can be further monitored as the cancer treatment progresses. In some embodiments, after a mammal that has been selected for further diagnostic testing has been administered a cancer treatment, the additional testing will reveal one or more cancer biomarkers. In some embodiments, such one or more cancer biomarkers (e.g., mutations) will provide cause to administer a different cancer treatment (e.g., a resistance mutation may arise in a cancer cell during the cancer treatment, which cancer cell harboring the resistance mutation is resistant to the original cancer treatment).

### Systems

In some examples, the present disclosure provides systems, methods, or kits that can include data analysis realized in measurement devices (e.g., laboratory instruments, such as a sequencing machine), software code that executes on computing hardware. The software can be stored in memory and execute on one or more hardware processors. The software can be organized into routines or packages that can communicate with each other. A module can comprise one or more devices/computers, and potentially one or more software routines/packages that execute on the one or more devices/computers. For example, an analysis application or system can include at least a data receiving module, a data pre-processing module, a data analysis module (which can operate on one or more types of genomic data), a data interpretation module, or a data visualization module.

The data receiving module can connect laboratory hardware or instrumentation with computer systems that process laboratory data. The data pre-processing module can perform operations on the data in preparation for analysis. Examples of operations that can be applied to the data in the pre-processing module include affine transformations, denoising operations, data cleaning, reformatting, or subsampling. The data analysis module, which can be specialized for analyzing genomic data from one or more genomic materials, can, for example, take assembled genomic sequences and perform probabilistic and statistical analysis to identify abnormal patterns related to a disease, pathology, state, risk, condition, or phenotype. The data interpretation module can use analysis methods, for example, drawn from statistics, mathematics, or biology, to support understanding of the relation between the identified abnormal patterns and health conditions, functional states, prognoses, or risks. The data analysis module and/or the data interpretation module can include one or more machine learning models, which can be implemented in hardware, e.g., which executes software that embodies a machine learning model. The data visualization module can use methods of mathematical modeling, computer graphics, or rendering to create visual representations of data that can facilitate the understanding or interpretation of results. The present disclosure provides computer systems that are programmed to implement methods of the disclosure.

In some embodiments, the methods disclosed herein can include computational analysis on nucleic acid sequencing data of samples from an individual or from a plurality of individuals. An analysis can identify a variant inferred from sequence data to identify sequence variants based on probabilistic modeling, statistical modeling, mechanistic modeling, network modeling, or statistical inferences. Non-limiting examples of analysis methods include principal component analysis, autoencoders, singular value decomposition, Fourier bases, wavelets, discriminant analysis, regression, support vector machines, tree-based methods, networks, matrix factorization, and clustering. Non-limiting examples of variants include a germline variation or a somatic mutation. In some examples, a variant can refer to an already-known variant. The already-known variant can be scientifically confirmed or reported in literature. In some examples, a variant can refer to a putative variant associated with a biological change. A biological change can be known or unknown. In some examples, a putative variant can be reported in literature, but not yet biologically confirmed. Alternatively, a putative variant is never reported in literature, but can be inferred based on a computational analysis disclosed herein. In some examples, germline variants can refer to nucleic acids that induce natural or normal variations.

In certain embodiments, the computer system includes a central processing unit (CPU, also “processor” and “computer processor” herein), which can be a single core or multi core processor, or a plurality of processors for parallel processing; memory (e.g., cache, random-access memory, read-only memory, flash memory, or other memory); electronic storage unit (e.g., hard disk), communication interface (e.g., network adapter) for communicating with one or more other systems; and peripheral devices, such as adapters for cache, other memory, data storage and/or electronic display. The memory, storage unit, interface and peripheral devices may be in communication with the CPU through a communication bus (solid lines), such as a motherboard. The storage unit can be a data storage unit (or data repository) for storing data. One or more analyte feature inputs can be entered from the one or more measurement devices. Example analytes and measurement devices are described herein.

The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing over the network (“the cloud”) to perform various aspects of analysis, calculation, and generation of the present disclosure, such as, for example, activation of a valve or pump to transfer a reagent or sample from one chamber to another or application of heat to a sample (e.g., during an amplification reaction), other aspects of processing and/or assaying a sample, performing sequencing analysis, measuring sets of values representative of classes of molecules, identifying sets of features and feature vectors from assay data, processing feature vectors using a machine learning model to obtain output classifications, and training a machine learning model (e.g., iteratively searching for optimal values of parameters of the machine learning model). Such cloud computing may be provided by cloud computing platforms such as, for example, Amazon Web Services (AWS), Microsoft Azure, Google Cloud Platform, and IBM cloud. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions can be stored in a memory location, such as the memory. The instructions can be directed to the CPU, which can subsequently program or otherwise configure the CPU to implement methods of the present disclosure. The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system via the network.

Methods as described herein can be implemented by way of machine (e.g., computer processor) executable code stored on an electronic storage location of the computer system such as, for example, on the memory or electronic storage unit. The machine executable or machine-readable code can be provided in the form of software. During use, the code can be executed by the CPU. In some cases, the code can be retrieved from the storage unit and stored on the memory for ready access by the CPU. In some situations, the electronic storage unit can be precluded, and machine-executable instructions are stored on memory.

The code can be pre-compiled and configured for use with a machine having a processer adapted to execute the code or can be compiled during runtime. The code can be supplied in a programming language that can be selected to enable the code to execute in a pre-compiled or as compiled fashion.

Aspects of the systems and methods provided herein, such as the computer system, can be embodied in programming. Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium, or physical transmission medium. Non-volatile storage media include, for example, optical or magnetic disks, such as any of the storage devices in any computer(s) or the like, such as can be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system.

Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media can be involved in carrying one or more sequences of one or more instructions to a processor for execution.

The computer system can include or be in communication with an electronic display that comprises a user interface (UI) for providing, for example, a current stage of processing or assaying of a sample (e.g., a particular step, such as a lysis step, or sequencing step that is being performed). Inputs are received by the computer system from one or more measurement. Examples of UIs include, without limitation, a graphical user interface (GUI) and web-based user interface. The algorithm can, for example, process and/or assay a sample, perform sequencing analysis, measure sets of values representative of classes of molecules, identify sets of features and feature vectors from assay data, process feature vectors using a machine learning model to obtain output classifications, and train a machine learning model (e.g., iteratively search for optimal values of parameters of the machine learning model).

In some embodiments, systems capable of executing one or more algorithms, e.g., laptops, desktops, iPads, mobile devices etc., for determining changes in cfDNA fragmentation profiles classifies the subject as a cancer patient based on the cfDNA fragmentation profile for the subject. These systems further execute machine learning algorithms that can be used to generate models such as, for example, high-risk populations and low-risk general populations (a penalized logistic regression with the Mathios et al. (Mathios D, Johansen J S, Cristiano S, Medina J E, Phallen J, Larsen K R, et al. Detection and characterization of lung cancer using cell-free DNA fragmentomes. Nat Commun 2021; 12(1):5060) features as well as coverage from transcription factor binding sites. These models can be trained on the subject cohort with 5-fold cross validation with 10 repeats, and scores for each sample ae calculated by the mean across repeats and evaluated using AUC-ROC. For example, the first model used the high-risk non-cancer and HCC patients while the second used the non-cancer individuals without liver pathology. The locked high-risk model trained on the cohort was applied to a second and different cohort to generate cancer predictions on an external validation set. A “class label” can be applied to each sample indicating the classification of the sample for any number of input features. For example, the class labels for the set of cohorts could indicate the identity of cfDNA fragmentation profiles based on genomic location etc. The resulting training sets are provided to machine learning unit, such as a neural network or a support vector machine. Using the training set, the machine learning unit may generate a model to classify the sample according to the cfDNA fragmentation profile.

In some embodiments, a method is provided for creating a trained classifier, comprising the steps of: (a) providing a plurality of different classes, wherein each class represents a set of subjects with a shared characteristic (e.g. from one or more cohorts); (b) providing a multi-parametric model representative of the cell-free DNA molecules from each of a plurality of samples belonging to each of the classes, thereby providing a training data set; and (c) training a learning algorithm on the training data set to create one or more trained classifiers, wherein each trained classifier classifies a test sample into one or more of the plurality of classes.

As an example, a trained classifier may use a learning algorithm selected from the group consisting of: a random forest, a neural network, a support vector machine, and a linear classifier. Each of the plurality of different classes may be selected from the group consisting of healthy, breast cancer, colon cancer, lung cancer, pancreatic cancer, prostate cancer, ovarian cancer, melanoma, and liver cancer.

A trained classifier may be applied to a method of classifying a sample from a subject. This method of classifying may comprise: (a) providing a multi-parametric model representative of the cell-free DNA molecules from a test sample from the subject; and (b) classifying the test sample using a trained classifier. After the test sample is classified into one or more classes, a therapeutic intervention on the subject can be performed based on the classification of the sample.

In some embodiments, training sets are provided to a machine learning unit, such as a neural network or a support vector machine. Using the training set, the machine learning unit may generate a model to classify the sample according to a treatment response to one or more therapeutic inventions. This is also referred to as “calling”. The model developed may employ information from any part of a test vector.

In general, machine learning can be used to reduce a set of data generated from all (primary sample/analytes/test) combinations into an optimal predictive set of features, e.g., which satisfy specified criteria. In various examples statistical learning, and/or regression analysis can be applied. Simple to complex and small to large models making a variety of modeling assumptions can be applied to the data in a cross-validation paradigm. Simple to complex includes considerations of linearity to non-linearity and non-hierarchical to hierarchical representations of the features. Small to large models includes considerations of the size of basis vector space to project the data onto as well as the number of interactions between features that are included in the modelling process.

Machine learning techniques can be used to assess the commercial testing modalities most optimal for cost/performance/commercial reach as defined in the initial question. A threshold check can be performed: If the method applied to a hold-out dataset that was not used in cross validation surpasses the initialized constraints, then the assay is locked, and production initiated. For example, a threshold for assay performance may include a desired minimum accuracy, positive predictive value (PPV), negative predictive value (NPV), clinical sensitivity, clinical specificity, area under the curve (AUC), or a combination thereof. For example, a desired minimum accuracy, PPV, NPV, clinical sensitivity, clinical specificity, or combination thereof may be at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. As another example, a desired minimum AUC may be at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99. A subset of assays may be selected from a set of assays to be performed on a given sample based on the total cost of performing the subset of assays, subject to the threshold for assay performance, such as desired minimum accuracy, positive predictive value (PPV), negative predictive value (NPV), clinical sensitivity, clinical specificity, area under the curve (AUC), and a combination thereof. If the thresholds are not met, then the assay engineering procedure can loop back to either the constraint setting for possible relaxation or to the wet lab to change the parameters in which data was acquired. Given the clinical question, biological constraints, budget, lab machines, etc., can constrain the problem.

In certain embodiments, the computer processing of a machine learning technique can include method(s) of statistics, mathematics, biology, or any combination thereof. In various examples, any one of the computer processing methods can include a dimension reduction method, logistic regression, dimension reduction, principal component analysis, autoencoders, singular value decomposition, Fourier bases, singular value decomposition, wavelets, discriminant analysis, support vector machine, tree-based methods, random forest, gradient boost tree, logistic regression, matrix factorization, network clustering, statistical testing and neural network.

In certain embodiments, the computer processing of a machine learning technique can include logistic regression, multiple linear regression (MLR), dimension reduction, partial least squares (PLS) regression, principal component regression, autoencoders, variational autoencoders, singular value decomposition, Fourier bases, wavelets, discriminant analysis, support vector machine, decision tree, classification and regression trees (CART), tree-based methods, random forest, gradient boost tree, logistic regression, matrix factorization, multidimensional scaling (MDS), dimensionality reduction methods, t-distributed stochastic neighbor embedding (t-SNE), multilayer perceptron (MLP), network clustering, neuro-fuzzy, neural networks (shallow and deep), artificial neural networks, Pearson product-moment correlation coefficient, Spearman's rank correlation coefficient, Kendall tau rank correlation coefficient, or any combination thereof. In some examples, the computer processing method is a supervised machine learning method including, for example, a regression, support vector machine, tree-based method, and neural network. In some examples, the computer processing method is an unsupervised machine learning method including, for example, clustering, network, principal component analysis, and matrix factorization.

For supervised learning, training samples (e.g., in thousands) can include measured data (e.g., of various analytes) and known labels, which may be determined via other time-consuming processes, such as imaging of the subject and analysis by a trained practitioner. Example labels can include classification of a subject, e.g., discrete classification of whether a subject has cancer or not or continuous classifications providing a probability (e.g., a risk or a score) of a discrete value. A learning module can optimize parameters of a model such that a quality metric (e.g., accuracy of prediction to known label) is achieved with one or more specified criteria. Determining a quality metric can be implemented for any arbitrary function including the set of all risk, loss, utility, and decision functions. A gradient can be used in conjunction with a learning step (e.g., a measure of how much the parameters of the model should be updated for a given time step of the optimization process).

As described above, examples can be used for a variety of purposes. For example, plasma (or other sample) can be collected from subjects symptomatic with a condition (e.g., known to have the condition) and healthy subjects. Genetic data (e.g., cfDNA) can be acquired analyzed to obtain a variety of different features, which can include features based on a genome wide analysis. These features can form a feature space that is searched, stretched, rotated, translated, and linearly or non-linearly transformed to generate an accurate machine learning model, which can differentiate between healthy subjects and subjects with the condition (e.g., identify a disease or non-disease status of a subject). Output derived from this data and model (which may include probabilities of the condition, stages (levels) of the condition, or other values), can be used to generate another model that can be used to recommend further procedures, e.g., recommend a biopsy or keep monitoring the subject condition.

In some embodiments, DNA from a population of several individuals can be analyzed by a set of multiplexed arrays. The data for each multiplexed array may be self-normalized using the information contained in that specific array. This normalization algorithm may adjust for nominal intensity variations observed in the two-color channels, background differences between the channels, and possible crosstalk between the dyes. The behavior of each base position may then be modeled using a clustering algorithm that incorporates several biological heuristics on fragmentation profiles. In cases where few cfDNA fragments are observed (e.g., due to low minor-allele frequency), locations and shapes of the missing sequences may be estimated using neural networks. Depending on the profiles and percent sequence identity, a statistical score may be devised (a Training score). A score such as GenCall Score is designed to mimic evaluations made by a human expert's visual and cognitive systems. In addition, it has been evolved using the genotyping data from top and bottom strands. This score may be combined with several penalty terms (e.g., low intensity, mismatch between existing and predicted cfDNA fragments) in order to make up the Training score. The Training score is saved for use by the calling algorithm.

To call a therapeutic response, a calling algorithm may take the genetic information and treatment responses of a plurality of individuals having a disease or condition. The data may first be normalized (using the same procedure as for the clustering algorithm). The calling operation (classification) may be performed using, for example, a Bayesian model. The score for each call's Call Score can be the product of a Training Score and a data-to-model fit score. After scoring all the treatment responses, the application may compute a composite score.

In some embodiments, a training dataset comprises clinical data selected from the group consisting of cancer stage, type of surgical procedure, age, tumor grading, depth of tumor infiltration, occurrence of post-operative complications, and the presence of venous invasion. In some embodiments, the training dataset is pre-processed, comprising transforming the provided data into class-conditional probabilities.

Another embodiment uses machine learning techniques to train a statistical classifier, specifically a support vector machine, for each cancer stage category based on word occurrences in a corpus of histology reports for each patient. New reports can then be classified according to the most likely stage, facilitating the collection and analysis of population staging data.

In some embodiments, a machine learning algorithm is selected from the group consisting of: a supervised or unsupervised learning algorithm selected from support vector machine, random forest, nearest neighbor analysis, linear regression, binary decision tree, discriminant analyses, logistic classifier, and cluster analysis.

In general, a system can comprise a report generator for reporting on cancer test results and treatment options. The report generator system can be a central data processing system configured to establish communications directly with: a remote data site or laboratory, a medical practice/healthcare provider (treating professional) and/or a patient/subject through communication links. The laboratory can be medical laboratory, diagnostic laboratory, medical facility, medical practice, point-of-care testing device, or any other remote data site capable of generating subject clinical information. Subject clinical information includes but it is not limited to laboratory test data, X-ray data, examination and diagnosis. The healthcare provider or practice 26 includes medical services providers, such as doctors, nurses, home health aides, technicians and physician's assistants, and the practice is any medical care facility staffed with healthcare providers. In certain instances, the healthcare provider/practice is also a remote data site. In a cancer treatment embodiment, the subject may be afflicted with cancer, among others.

Other clinical information for a cancer subject includes the results of laboratory tests, imaging or medical procedure directed towards the specific cancer that one of ordinary skill in the art can readily identify. The list of appropriate sources of clinical information for cancer includes but it is not limited to: CT scan, MRI scan, ultrasound scan, bone scan, PET Scan, bone marrow test, barium X-ray, endoscopy, lymphangiogram, IVU (Intravenous urogram) or IVP (IV pyelogram), lumbar puncture, cystoscopy, immunological tests (anti-malignin antibody screen), and cancer marker tests.

The subject clinical information may be obtained from the laboratory manually or automatically. For simplicity of the system the information is obtained automatically at predetermined or regular time intervals. A regular time interval refers to a time interval at which the collection of the laboratory data is carried out automatically by the methods and systems described herein based on a measurement of time such as hours, days, weeks, months, years etc. In one embodiment of the invention, the collection of data and processing is carried out at least once a day. In one embodiment, the transfer and collection of data is carried out once every month, biweekly, or once a week, or once every couple of days. Alternatively, the retrieval of information may be carried out at predetermined but not regular time intervals. For instance, a first retrieval step may occur after one week and a second retrieval step may occur after one month. The transfer and collection of data can be customized according to the nature of the disorder that is being managed and the frequency of required testing and medical examinations of the subjects.

In certain embodiments, a genetic report is generated from a subject's sample, e.g. cfDNA. The polynucleotides in a sample can be sequenced, e.g., whole genome sequencing, NGS sequencing, producing a plurality of sequence reads. In some embodiments, genetic information comprises variables defining the genomic organization of cancer cells or the genomic organization of single disseminated cancer cells. In some embodiments, the genetic information comprises sequence or abundance data from one or more genetic loci in cell-free DNA from the individuals.

cfDNA genetic information is processed (72). Genetic variants can also be identified. Genetic variants include sequence variants, copy number variants and nucleotide modification variants. A sequence variant is a variation in a genetic nucleotide sequence. A copy number variant is a deviation from wild type in the number of copies of a portion of a genome. Genetic variants include, for example, single nucleotide variations (SNPs), insertions, deletions, inversions, transversions, translocations, gene fusions, chromosome fusions, gene truncations, copy number variations (e.g., aneuploidy, partial aneuploidy, polyploidy, gene amplification), abnormal changes in nucleic acid chemical modifications, abnormal changes in epigenetic patterns and abnormal changes in nucleic acid methylation. The process then determines the frequency of genetic variants in the sample containing the genetic material. Since this process is noisy, the process separates information from noise (73). The sensitivity of detecting genetic variants can be increased by increasing read depth of polynucleotides (e.g., by sequencing to a greater read depth at in a sample from a subject at two or more time points).

To increase the diagnosis confidence, a plurality of measurements can be taken. Or alternatively using measurements at a plurality of time points (e.g., 2, 3, 4, 5, 6, 7, 8, 9, 10, or more time points) to determine whether cancer is advancing, in remission or stabilized. The diagnostic confidence can be used to identify disease states. For example, cell free polynucleotides taken from a subject can include polynucleotides derived from normal cells, as well as polynucleotides derived from diseased cells, such as cancer cells. Polynucleotides from cancer cells may bear genetic variants, such as somatic cell mutations and copy number variants. When cell free polynucleotides from a sample from a subject are sequenced, and cfDNA fragmentation profiles can be produced as described in the examples section which follows.

Numerous cancers may be detected using the methods and systems described herein. Cancers cells, as most cells, can be characterized by a rate of turnover, in which old cells die and replaced by newer cells. Generally dead cells, in contact with vasculature in a given subject, may release DNA or fragments of DNA into the blood stream. This is also true of cancer cells during various stages of the disease. Cancer cells may also be characterized, dependent on the stage of the disease, by various genetic aberrations such as copy number variation as well as mutations. This phenomenon may be used to detect the presence or absence of cancers individuals using the methods and systems described herein.

In the early detection of cancers, any of the systems or methods herein described, including mutation detection or copy number variation detection may be utilized to detect cancers. These system and methods may be used to detect any number of genetic aberrations that may cause or result from cancers. These may include but are not limited to cfDNA fragmentation profiles, mutations, mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, abnormal changes in nucleic acid methylation infection and cancer.

Additionally, the systems and methods described herein may also be used to help characterize certain cancers. Genetic data produced from the system and methods of this disclosure may allow practitioners to help better characterize a specific form of cancer. Often times, cancers are heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer.

The systems and methods provided herein may be used to monitor already known cancers, or other diseases in a particular subject. This may allow either a subject or practitioner to adapt treatment options in accord with the progress of the disease. In this example, the systems and methods described herein may be used to construct genetic cfDNA fragmentation profiles of a particular subject of the course of the disease. In some instances, cancers can progress, becoming more aggressive and genetically unstable. In other examples, cancers may remain benign, inactive or dormant. The system and methods of this disclosure may be useful in determining disease progression.

Further, the systems and methods described herein may be useful in determining the efficacy of a particular treatment option. In one example, certain treatment options may be correlated with genetic cfDNA fragmentation profiles of cancers over time. This correlation may be useful in selecting a therapy. Additionally, if a cancer is observed to be in remission after treatment, the systems and methods described herein may be useful in monitoring residual disease or recurrence of disease.

Further, the methods of the disclosure may be used to characterize the heterogeneity of an abnormal condition in a subject, the method comprising generating a cfDNA fragmentation profile of extracellular polynucleotides in the subject, wherein the cfDNA fragmentation profile comprises a plurality of data resulting from profile variation and mutation analyses. In some cases, including but not limited to cancer, a disease may be heterogeneous. Disease cells may not be identical. In the example of cancer, some tumors are known to comprise different types of tumor cells, some cells in different stages of the cancer. In other examples, heterogeneity may comprise multiple foci of disease. Again, in the example of cancer, there may be multiple tumor foci, perhaps where one or more foci are the result of metastases that have spread from a primary site (also known as distant metastases).

The methods of this disclosure may be used to generate a profile, fingerprint, or set of data that is a summation of genetic information derived from different cells in a heterogeneous disease. This set of data may comprise copy number variation and mutation analyses alone or in combination.

Further, these reports are submitted and accessed electronically via the internet. Analysis of data occurs at a site other than the location of the subject. The report is generated and transmitted to the subject's location. Via an internet enabled computer, the subject accesses the reports reflecting his tumor burden.

The annotated information can be used by a health care provider to select other drug treatment options and/or provide information about drug treatment options to an insurance company. The method can include annotating the drug treatment options for a condition in, for example, the NCCN Clinical Practice Guidelines in Oncology™ or the American Society of Clinical Oncology (ASCO) clinical practice guidelines.

Reports are generated, mapping genome positions and cfDNA fragmentation profile variation for the subject with cancer. These reports, in comparison to other profiles of subjects with known outcomes, can indicate that a particular cancer is aggressive and resistant to treatment. The subject is monitored for a period and retested. If at the end of the period, the cfDNA fragmentation variation profile does not vary, this may indicate that the current treatment is not working. A comparison is done with cfDNA fragmentation profiles of other subjects. For example, if it is determined that a change in cfDNA fragmentation variation indicates that the cancer is advancing, then the original treatment regimen as prescribed is no longer treating the cancer and a new treatment is prescribed.

In certain embodiments, the system receives genetic information from a DNA sequencer. The process then determines specific cfDNA fragmentation alterations and quantities thereof. These reports are submitted and accessed electronically via the internet. Analysis of data occurs at a site other than the location of the subject. The report is generated and transmitted to the subject's location. Via an internet enabled computer, the subject accesses the reports reflecting his tumor burden.

While temporal information can be used to enhance the information for cfDNA fragmentation profiles, other consensus methods can be applied. In other embodiments, the historical comparison can be used in conjunction with other consensus cfDNA fragmentation profiles. Consensus cfDNA fragmentation profiles can be normalized against control samples. Measures of molecules mapping to reference sequences can also be compared across a genome to identify areas in the genome in which cfDNA fragmentation profiles varies, or remains the same. Consensus methods include, for example, linear or non-linear methods of building consensus cfDNA fragmentation profiles (such as voting, averaging, statistical, maximum a posteriori or maximum likelihood detection, dynamic programming, Bayesian, hidden Markov or support vector machine methods, etc.) derived from digital communication theory, information theory, or bioinformatics. After the sequence read coverage has been determined, a stochastic modeling algorithm is applied to convert the normalized nucleic acid sequence read coverage for each window region to the discrete copy number states. In some cases, this algorithm may comprise one or more of the following: Hidden Markov Model, dynamic programming, support vector machine, Bayesian network, trellis decoding, Viterbi decoding, expectation maximization, Kalman filtering methodologies and neural networks.

Artificial neural networks (NNets) mimic networks of “neurons” based on the neural structure of the brain. They process records one at a time, or in a batch mode, and “learn” by comparing their classification of the record (which, at the outset, is largely arbitrary) with the known actual classification of the record. In MLP-NNets, the errors from the initial classification of the first record is fed back into the network, and are used to modify the network's algorithm the second time around, and so on for many iterations. The neural networks use an iterative learning process in which data cases (rows) are presented to the network one at a time, and the weights associated with the input values are adjusted each time.

After all cases are presented, the process often starts over again. During this learning phase, the network learns by adjusting the weights so as to be able to predict the correct class label of input samples. Neural network learning is also referred to as “connectionist learning,” due to connections between the units. Advantages of neural networks include their high tolerance to noisy data, as well as their ability to classify patterns on which they have not been trained. One neural network algorithm is back-propagation algorithm, such as Levenberg-Marquadt. Once a network has been structured for a particular application, that network is ready to be trained. To start this process, the initial weights are chosen randomly. Then the training, or learning, begins.

The network processes the records in the training data one at a time, using the weights and functions in the hidden layers, then compares the resulting outputs against the desired outputs. Errors are then propagated back through the system, causing the system to adjust the weights for application to the next record to be processed. This process occurs over and over as the weights are continually tweaked. During the training of a network the same set of data is processed many times as the connection weights are continually refined.

In an embodiment, the training step of the machine learning unit on the training data set may generate one or more classification models for applying to a test sample. These classification models may be applied to a test sample to predict the response of a subject to a therapeutic intervention.

Comparison of sequence coverage to a control sample or reference sequence may aid in normalization across windows. In this embodiment, cell free DNAs are extracted and isolated from a readily accessible bodily fluid such as blood. For example, cell free DNAs can be extracted using a variety of methods known in the art, including but not limited to isopropanol precipitation and/or silica based purification. Cell free DNAs may be extracted from any number of subjects, such as subjects without cancer, subjects at risk for cancer, or subjects known to have cancer (e.g. through other means).

Following the isolation/extraction step, any of a number of different sequencing operations may be performed on the cell free polynucleotide sample. Samples may be processed before sequencing with one or more reagents (e.g., enzymes, unique identifiers (e.g., barcodes), probes, etc.). In some cases if the sample is processed with a unique identifier such as a barcode, the samples or fragments of samples may be tagged individually or in subgroups with the unique identifier. The tagged sample may then be used in a downstream application such as a sequencing reaction by which individual molecules may be tracked to parent molecules.

The cell free polynucleotides can be tagged or tracked in order to permit subsequent identification and origin of the particular polynucleotide. The assignment of an identifier (e.g., a barcode) to individual or subgroups of polynucleotides may allow for a unique identity to be assigned to individual sequences or fragments of sequences. This may allow acquisition of data from individual samples and is not limited to averages of samples. In some examples, nucleic acids or other molecules derived from a single strand may share a common tag or identifier and therefore may be later identified as being derived from that strand. Similarly, all of the fragments from a single strand of nucleic acid may be tagged with the same identifier or tag, thereby permitting subsequent identification of fragments from the parent strand. In other cases, gene expression products (e.g., mRNA) may be tagged in order to quantify expression, by which the barcode, or the barcode in combination with sequence to which it is attached can be counted. In still other cases, the systems and methods can be used as a PCR amplification control. In such cases, multiple amplification products from a PCR reaction can be tagged with the same tag or identifier. If the products are later sequenced and demonstrate sequence differences, differences among products with the same identifier can then be attributed to PCR error. Additionally, individual sequences may be identified based upon characteristics of sequence data for the read themselves. For example, the detection of unique sequence data at the beginning (start) and end (stop) portions of individual sequencing reads may be used, alone or in combination, with the length, or number of base pairs of each sequence read unique sequence to assign unique identities to individual molecules. Fragments from a single strand of nucleic acid, having been assigned a unique identity, may thereby permit subsequent identification of fragments from the parent strand. This can be used in conjunction with bottlenecking the initial starting genetic material to limit diversity.

Generally, the methods and systems provided herein are useful for preparation of cell free polynucleotide sequences to a down-stream application sequencing reaction. Often, a sequencing method is next generation sequencing (NGS), classic Sanger sequencing, whole-genome bisulfite sequencing (WGSB), small-RNA sequencing, low-coverage Whole-Genome Sequencing (lcWGS), etc.

As used herein, the term “sequencing” refers to any of a number of technologies used to determine the sequence of a biomolecule, e.g., a nucleic acid such as DNA or RNA. Exemplary sequencing methods include, but are not limited to, targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performer by a gene analyzer such as, for example, gene analyzers commercially available from Illumina or Applied Biosystems. In some embodiments, the sequencing method can be massively parallel sequencing, that is, simultaneously (or in rapid succession) sequencing any of at least 100, 1000, 10,000, 100,000, 1 million, 10 million, 100 million, or 1 billion polynucleotide molecules.

After sequencing, reads are assigned a quality score. A quality score may be a representation of reads that indicates whether those reads may be useful in subsequent analysis based on a threshold. In some cases, some reads are not of sufficient quality or length to perform the subsequent mapping step. Sequencing reads with a quality score at least 90%, 95%, 99%, 99.9%, 99.99% or 99.999% may be filtered out of the data set. In other cases, sequencing reads assigned a quality scored at least 90%, 95%, 99%, 99.9%, 99.99% or 99.999% may be filtered out of the data set. In step 306, the genomic fragment reads that meet a specified quality score threshold are mapped to a reference genome, or a reference sequence that is known not to contain mutations. After mapping alignment, sequence reads are assigned a mapping score. A mapping score may be a representation or reads mapped back to the reference sequence indicating whether each position is or is not uniquely mappable. In instances, reads may be sequences unrelated to mutation analysis. For example, some sequence reads may originate from contaminant polynucleotides. Sequencing reads with a mapping score at least 90%, 95%, 99%, 99.9%, 99.99% or 99.999% may be filtered out of the data set. In other cases, sequencing reads assigned a mapping scored less than 90%, 95%, 99%, 99.9%, 99.99% or 99.999% may be filtered out of the data set. For each mappable base, bases that do not meet the minimum threshold for mappability, or low quality bases, may be replaced by the corresponding bases as found in the reference sequence.

Numerous cancers may be detected using the methods and systems described herein. Cancers cells, as most cells, can be characterized by a rate of turnover, in which old cells die and replaced by newer cells. Generally dead cells, in contact with vasculature in a given subject, may release DNA or fragments of DNA into the blood stream. This is also true of cancer cells during various stages of the disease. Cancer cells may also be characterized, dependent on the stage of the disease, by various genetic aberrations such as copy number variation as well as mutations. This phenomenon may be used to detect the presence or absence of cancers individuals using the methods and systems described herein.

The types and number of cancers that may be detected may include but are not limited to blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like.

Additionally, the systems and methods described herein may also be used to help characterize certain cancers. Genetic data produced from the system and methods of this disclosure may allow practitioners to help better characterize a specific form of cancer. Often times, cancers are heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer.

The systems and methods provided herein may be used to monitor already known cancers, or other diseases in a particular subject. This may allow either a subject or practitioner to adapt treatment options in accord with the progress of the disease. In this example, the systems and methods described herein may be used to construct genetic profiles of a particular subject of the course of the disease. In some instances, cancers can progress, becoming more aggressive and genetically unstable. In other examples, cancers may remain benign, inactive or dormant. The system and methods of this disclosure may be useful in determining disease progression.

Further, the systems and methods described herein may be useful in determining the efficacy of a particular treatment option. In one example, successful treatment options may actually increase the amount of copy number variation or mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the systems and methods described herein may be useful in monitoring residual disease or recurrence of disease.

The data is sent over a direct connection or over the internet to a computer for processing. The data processing aspects of the system can be implemented in digital electronic circuitry, or in computer hardware, firmware, software, or in combinations of them. Data processing apparatus of the invention can be implemented in a computer program product tangibly embodied in a machine-readable storage device for execution by a programmable processor; and data processing method steps of the invention can be performed by a programmable processor executing a program of instructions to perform functions of the invention by operating on input data and generating output. The data processing aspects of the invention can be implemented advantageously in one or more computer programs that are executable on a programmable system including at least one programmable processor coupled to receive data and instructions from and to transmit data and instructions to a data storage system, at least one input device, and at least one output device. Each computer program can be implemented in a high-level procedural or object-oriented programming language, or in assembly or machine language, if desired; and, in any case, the language can be a compiled or interpreted language. Suitable processors include, by way of example, both general and special purpose microprocessors. Generally, a processor will receive instructions and data from a read-only memory and/or a random access memory. Storage devices suitable for tangibly embodying computer program instructions and data include all forms of nonvolatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs (application-specific integrated circuits).

To provide for interaction with a user, the methods can be implemented using a computer system having a display device such as a monitor or LCD (liquid crystal display) screen for displaying information to the user and input devices by which the user can provide input to the computer system such as a keyboard, a two-dimensional pointing device such as a mouse or a trackball, or a three-dimensional pointing device such as a data glove or a gyroscopic mouse. The computer system can be programmed to provide a graphical user interface through which computer programs interact with users. The computer system can be programmed to provide a virtual reality, three-dimensional display interface.

### EXAMPLES

### Example 1: Clinical Cohorts and Genomic Analyses of cfDNA

We examined plasma samples from 501 individuals, including 75 individuals with HCC and 426 without cancer. Among individuals without cancer, 133 had conditions that increased HCC risk, including cirrhosis from all causes or viral hepatitis without cirrhosis. Blood samples were prospectively collected from patients with HCC at various cancer stages and from high-risk individuals at the Johns Hopkins Hospital, while the remaining samples were identified through screening efforts at other US or EU hospitals (US/EU cohort) (Table 1). We isolated 0.5 to 5 ml of plasma from each of these individuals, generated genomic libraries, and sequenced the cfDNA fragments using low coverage whole genome sequencing (~2.6× coverage) with an average of 49 million high quality paired reads per sample comprising 9 Gb of sequence data (24,25). In addition to the US/EU cohort, we examined as a validation cohort whole genome sequence data from 223 patients from Hong Kong, including patients with resectable early-stage HCC (n=90, stage A=85, B=5), HBV (n=66) and HBV-related cirrhosis (n=35), as well as healthy individuals without liver disease (n=32) (Hong Kong cohort) (Table 1) (15,28).

Genome-Wide cfDNA Fragmentation Profiles Informed by Underlying Chromatin Structure

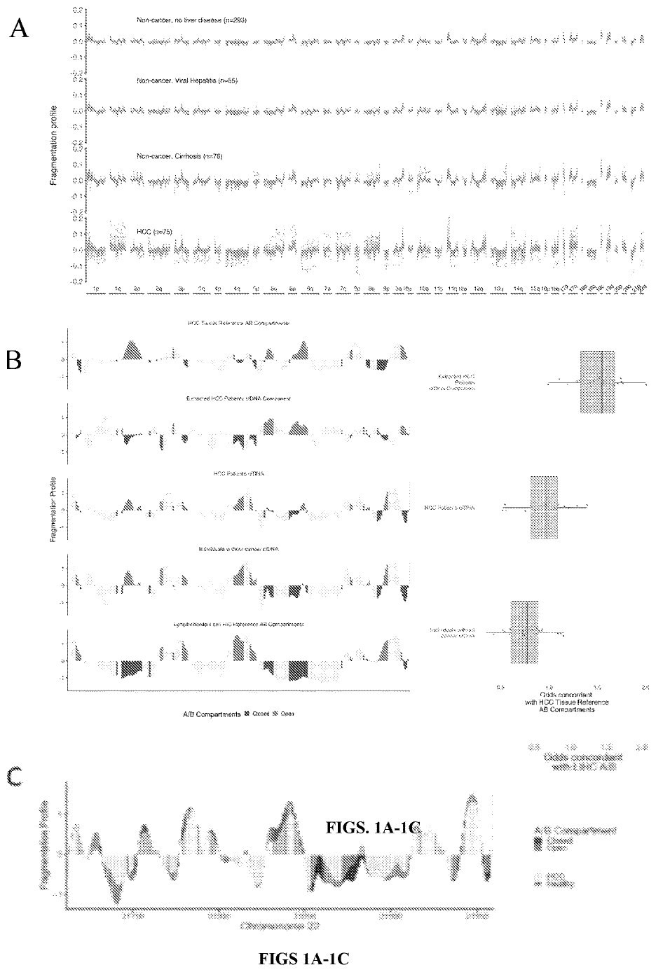

We evaluated the fragmentome and generated fragmentation profiles across the genome in 473 non-overlapping 5 MB regions, each region comprising ~80,000 fragments, and spanning approximately 2.4 GB of the genome using the DELFI approach (24). The fragmentation profiles were consistent among individuals without cancer, but highly variable among patients with HCC (FIG. 1A). Profiles of patients with cirrhosis were closer to non-cancer individuals without cirrhosis than they were to those from patients with HCC (FIG. 1A). Likewise, patients with viral hepatitis had fragmentation profiles nearly identical to those of non-cancer individuals without liver disease (FIG. 1A).

To examine the origins of cfDNA fragmentation patterns, we compared genome-wide fragmentome profiles to high-throughput sequencing chromosome conformation capture (Hi-C) open (A) and closed (B) compartments. We found that cfDNA patterns of healthy individuals were highly correlated to those of lymphoblastoid cells (FIG. 1B). Analysis of cfDNA profiles from 10 HCC patients with high ctDNA levels revealed that their fragmentome reflected two components, one resembling the profile of individuals without cancer, as well as a separate cfDNA component that had high similarity to A/B compartments previously estimated from liver cancers (FIG. 1B) (29). Additionally, when these two components were estimated, the cfDNA profiles of the predicted liver component had high similarity to genome-wide A/B compartments of liver cancer, while the profiles of patients with HCC were intermediate in similarity to liver cancer (FIG. 1B, C). In contrast, the profiles of individuals without cancer were closer to A/B compartments of lymphoblastoid cells (FIG. 1B, 1C). These analyses suggested that cfDNA fragmentomes from individuals with HCC represent a mixture of cfDNA profiles of chromatin compartments of cells from peripheral blood as well as those from liver cancer.

Disease-Specific Transcription Factors Inferred from Genome-Wide cfDNA Fragmentation

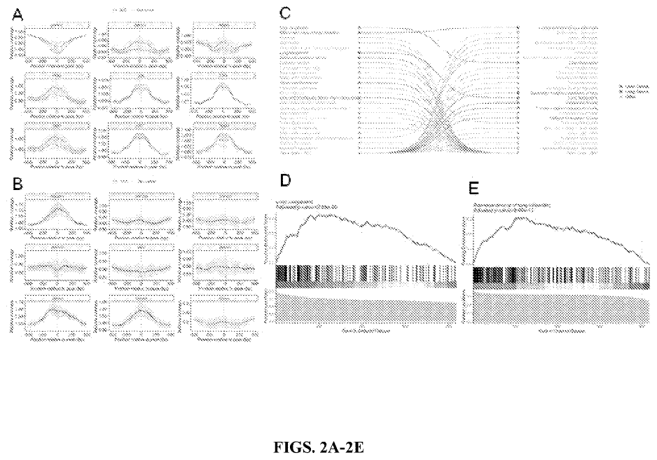

As chromatin organization reflects underlying cellular transcriptional programs (30-32), we examined whether cfDNA fragmentation characteristics might reflect changes derived from altered DNA binding of transcription factors (TFs) in liver cancer. To identify DNA binding sites for all known TFs, we analyzed 5620 CHIP-seq experiments from the ReMap 2020 database (33). For each TF, we calculated the aggregate cfDNA coverage across all binding sites identified (4K-490K per sample) compared to the overall adjacent genomic coverage, producing a single metric for each TF in each sample. We compared these TFs in patients with and without HCC to identify those TFs with the largest and smallest differences in genome-wide binding site coverage in cfDNA (FIG. 2A, B). Gene set enrichment analyses using the DisGeNET database of gene-disease associations revealed that differences in cfDNA TF binding coverages between HCC and individuals without cancer were predicted to be related to liver and other cancers (FIG. 2C, D). Additionally, the top scoring individual TFs represented those with known biological relevance to chromatin organization and liver cancer (Table 2). These included members of the of the activator protein 1 (AP1) complex, including JUN, JUND, ATF2, and ATF7 genes, which integrate extracellular signals (34) and have been linked to liver tumorigenesis (35,36), Transcriptional Enhancer Factor Domain Family member 4 (TEAD4) which has been shown to have oncogenic roles in HCC (37,38), Poly(C)-binding protein 2 (PCBP2) transcriptional coregulator which when overexpressed is associated with a worse prognosis in patients with HCC (39), Prohibitin 2 (PHB), which promotes progression in HCC (40), and AT-rich interacting domain 3A (ARID3A), an oncogenic transcription factor that when upregulated promotes liver cancer malignancy (41). A similar analysis of cfDNA fragmentation data from our recent study of patients in the LUCAS lung cancer diagnostic trial (24) revealed an enrichment of coverage differences in binding sites of TFs related to lung cancer (FIG. 2C, E). Altogether, these observations suggest that changes in cfDNA fragmentation in patients with liver and other cancers result from the multitude of altered transcriptional profiles present in the cancer cells.

Genomic Changes in HCC are Revealed from cfDNA Fragmentomes

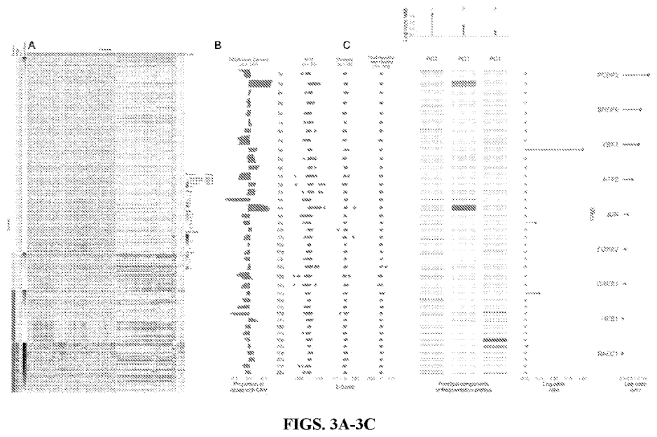

As the cfDNA fragmentome may comprise changes related to large-scale genomic alterations released from cancer cells (24,25), we also examined chromosomal gains and losses in the circulation of these patients. In addition to the genome-wide fragmentation profiles resulting from chromatin and transcription factor changes observed in patients with liver cancer (FIG. 3A), our analyses revealed an altered representation of chromosomal arms matching those commonly gained or lost in liver cancer as reported in previous TCGA large-scale genomic studies of HCC (n=372) (FIG. 3B). These included increased cfDNA representation of 1q, 7p, 7q, 8q and decreased levels of 4q, 8p, 9p, 13q, and 21q, all known to be gained or lost, respectively, in HCC (42,43). Importantly, these alterations were observed in the patients with HCC but not in individuals without cancer, even if they had cirrhosis or chronic liver disease (FIG. 3B).

### DELFI Model for HCC Detection

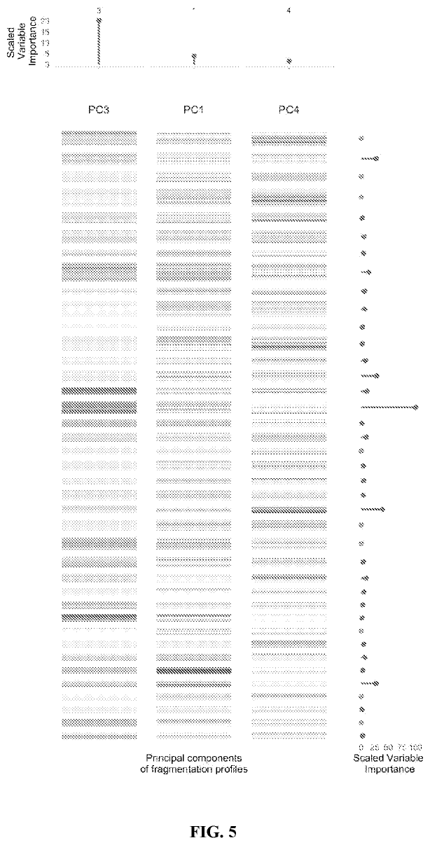

Given the direct connection between genomic and chromatin changes in liver cancer and cfDNA fragmentation, we used a machine learning approach to determine if changes in cfDNA fragmentomes could distinguish patients with HCC from those without cancer. We previously used this approach to develop a robust classifier for lung cancer detection that was externally validated in an independent population (24). We determined the performance of this classifier in the US/EU cohort by repeated fivefold cross-validation, generating a score for each individual that is an average over ten cross-validation repeats (DELFI score). The resulting model included a combination of regional and large-scale fragmentation characteristics that were optimal for identifying individuals with liver cancer (FIG. 5). These features comprised the majority of informative chromosomal, chromatin, and local changes identified above, comprising >90% of the variance of the fragmentation profiles across samples.

As clinical characteristics may affect tumor biomarkers, we investigated whether measures of liver dysfunction or demographic parameters such as age, sex, race, or weight were associated with DELFI scores in individuals without cancer where this information was available.

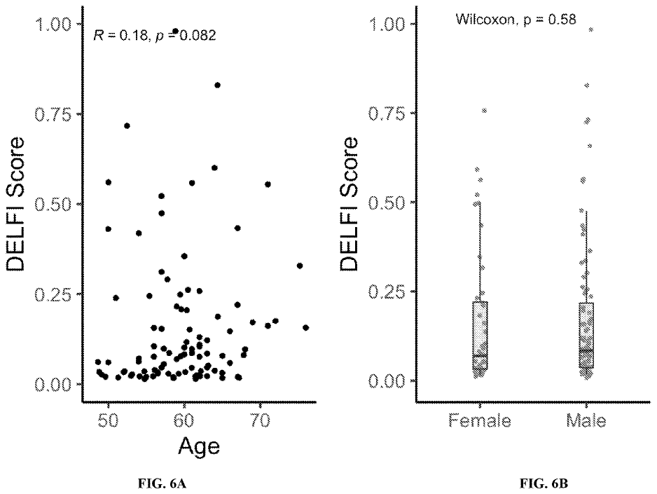

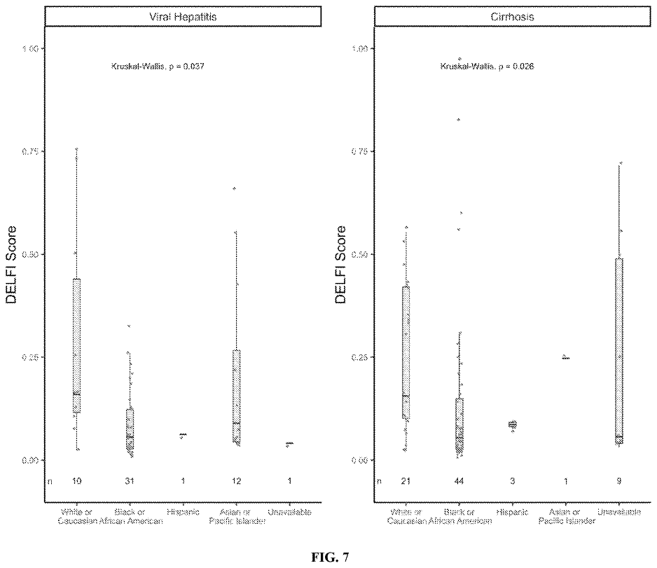

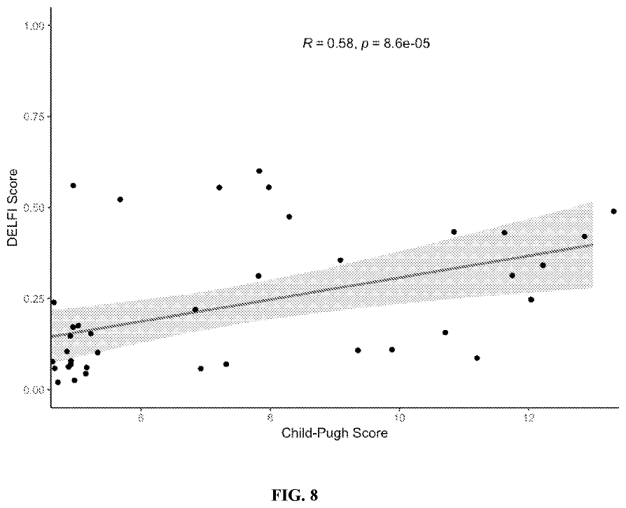

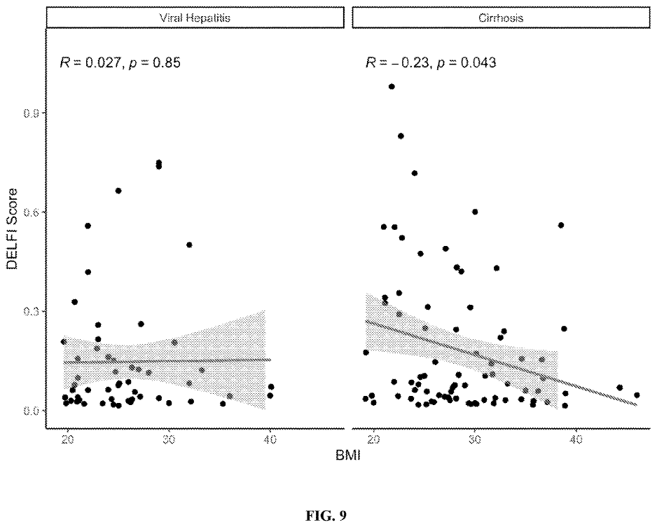

We observed no association of DELFI scores with age (R=0.18, p=0.08, Spearman correlation) (FIG. 6A) and no difference in DELFI scores between males and females (p=0.58, Wilcoxon test) (FIG. 6B). Asians and African-Americans have been shown to have a higher incidence of liver cancer which is diagnosed at later stages (44), and we observed small differences in fragmentation scores among high-risk individuals without cancer across these or other racial or ethnic groups, although these analyses are limited by lack of information on clinical covariates in some of these cases (p=0.037 in patients with viral hepatitis, and p=0.026 in cirrhotic patients, Kruskal-Wallis test) (FIG. 7). Among individuals with cirrhosis, we observed a correlation between the degree of liver disease as measured by Child-Pugh Score and DELFI scores (R=0.58 p=8.6e-5, Spearman correlation) (FIG. 8). Increased body mass index (BMI), a risk factor for NAFLD and liver cancer, was not associated with changes in DELFI scores in patients with viral hepatitis (R=0.027, p=0.85, Spearman correlation); however, lower BMI in cirrhotic patients was associated with higher DELFI scores, perhaps due to cachexia in patients with severe cirrhosis (R=−0.23, p=0.043, Spearman correlation) (FIG. 9).

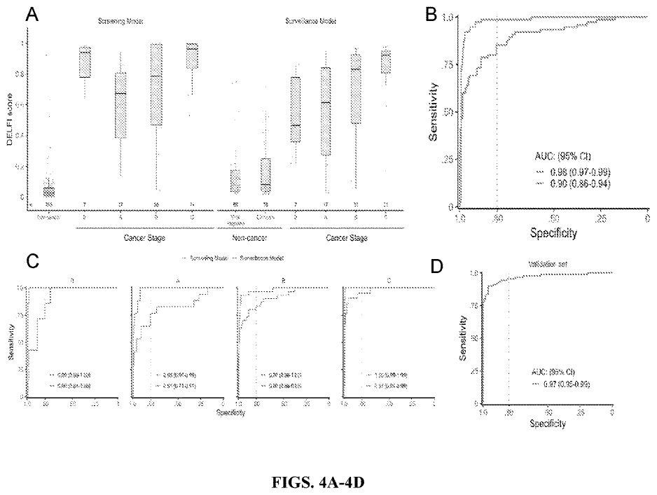

We next examined the relationship between DELFI scores and the presence and stage of liver cancer in a population at high risk for liver cancer. The DELFI scores for 133 individuals who were cancer-free were low, with median DELFI scores of 0.078 or 0.080 for those with viral hepatitis or cirrhosis, respectively. In contrast, the 75 patients with HCC had significantly higher median DELFI scores across all BCLC stages, including stage 0=0.46, stage A=0.61, stage B=0.83, and stage C=0.92 (p<0.01 for Stages 0, A, B, or C, Wilcoxon rank sum test, FIG. 4A). A receiver operator characteristic (ROC) curve of the DELFI approach to identify HCC patients revealed an area under the curve (AUC) of 0.90 (95% CI=0.86-0.94) among high-risk individuals. Performance remained robust for early stage HCCs with AUCs of 0.9 and 0.81 for BCLC stage 0 and A. Advanced-stage HCC (BCLC C) individuals were almost perfectly detected among the individuals analyzed (AUC>0.97) (FIG. 4C).

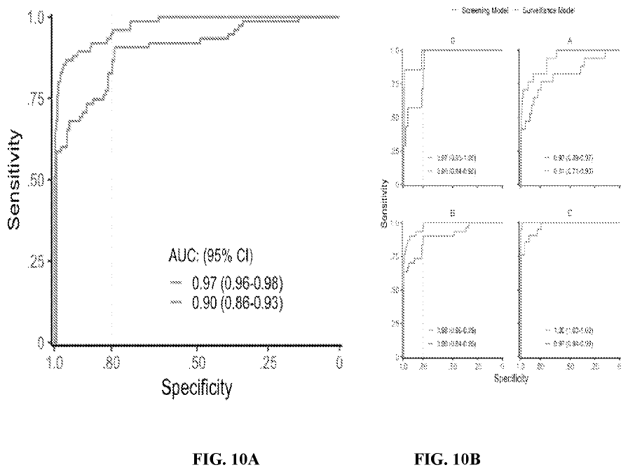

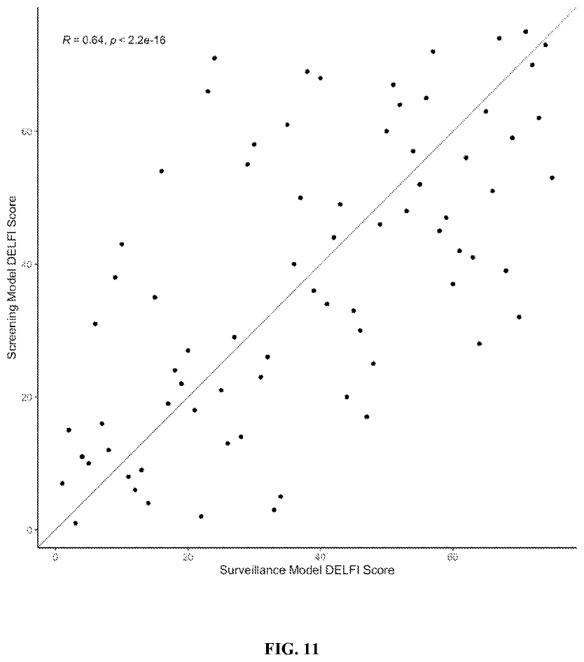

To extend these analyses to individuals at low risk for developing liver cancer, we examined the ability of a DELFI model to distinguish between individuals with cancer and those from a general population (n=293) without viral hepatitis or cirrhosis. In this larger cohort where additional features could be included in cross validated training, we used the features of the model above and also included cfDNA coverage at CHIP-seq derived transcription factor binding sites from liver cell lines available in the ReMAP database to create a DELFI model for a general population. This approach had high performance for cancer detection (AUC=0.98) among these individuals. We evaluated the performance of this model at 99% specificity, an threshold appropriate for an average risk population (25), and observed an overall sensitivity of 80% in this setting (FIG. 4B), with sensitivity above 65% across all stages. Use of a model that did not incorporate transcription factor binding sites led to a slightly reduced performance, and there was high correlation among the rank ordered scores using our DELFI models for high risk and screening populations (R=0.64, p<1e-15) (FIGS. 10 and 11).

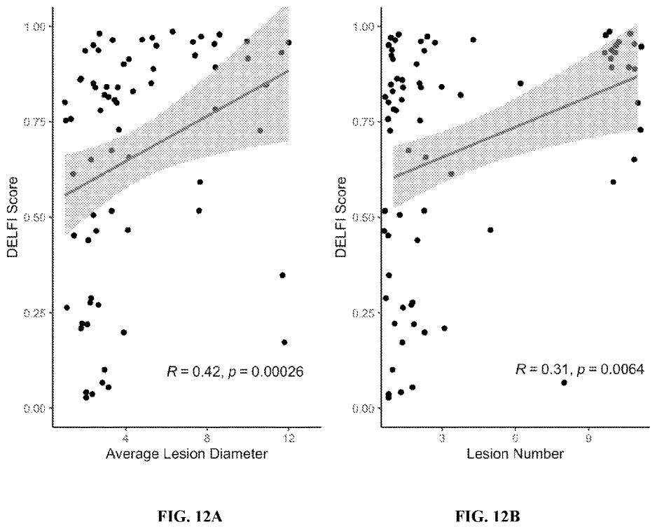

To examine the relationship between fragmentation profiles and liver cancer progression, we assessed whether the size, number, and characteristics of liver cancer lesions as well as the etiology of neoplasia was related to aberrant fragmentation profiles, where this information was available. We found that the tumor size and lesion number were positively correlated to DELFI scores (R=0.42 and 0.31, p=0.00026 and p=0.0064 respectively, Spearman correlation) (FIG. 12), consistent with the notion that the fragmentation profile was related to overall tumor burden.

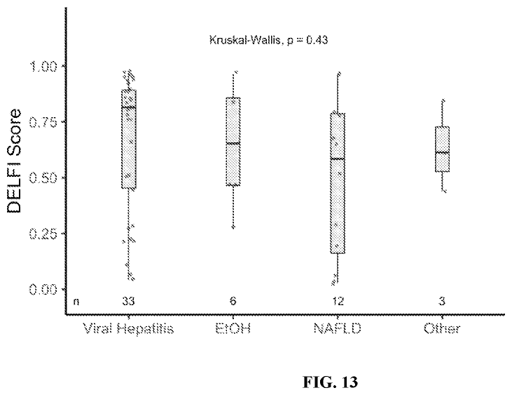

Among patients with liver cancer at resectable stages (0, A, and B), the cancer etiology, including viral hepatitis, or cirrhosis due to alcohol, NAFLD, or idiopathic sources, yielded similar DELFI scores (p=0.43, Kruskal-Wallis test) (FIG. 13). These observations suggest that fragmentation profiles were a result of ongoing tumor-related cfDNA processes and were not affected by early events in tumorigenesis.

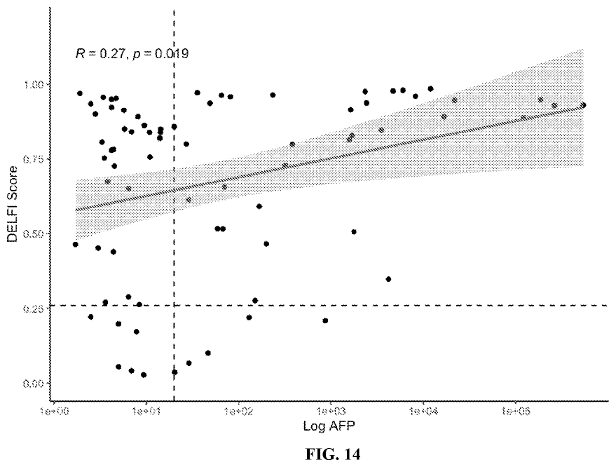

To examine the real-world impact of this method in the context of HCC detection, we compared the performance of DELFI fragmentome performance to the current screening measurement of alpha-fetoprotein protein (AFP) levels. AFP levels were elevated above the recommended screening threshold of 20 ng/ml in 39 of 75 (52%) individuals with cancer, consistent with previous reports (45). Among individuals that had AFP levels below 20 ng/ml and who have been undetected by this approach, DELFI detected 30 of 36 (83%). The use of AFP measurements would have detected 8/24 (33%) stage 0/A patients, 17/30 (57%) of stage B patients, and 14/21 (66%) stage C patients (FIG. 14). In contrast, the DELFI approach detected 19/24 (79%) stage 0/A patients, 25/30 (83%) stage B patients, and 20/21 (95%) stage C patients. Overall, genome-wide cfDNA fragmentation analyses had improved performance compared to AFP detection of HCC, and the combination of DELFI and AFP may provide an improvement in detection over the DELFI approach alone, as we observed these to have a combined sensitivity of 92% at a combined specificity of 80%.

External Validation of DELFI Model in East Asian Population with HCC

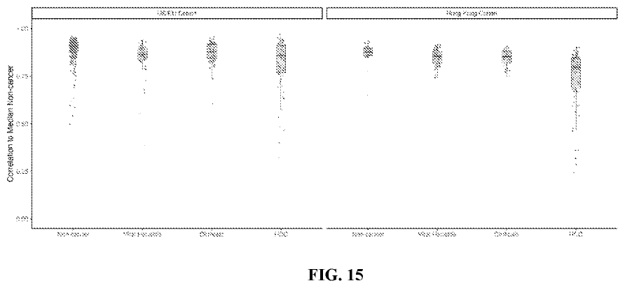

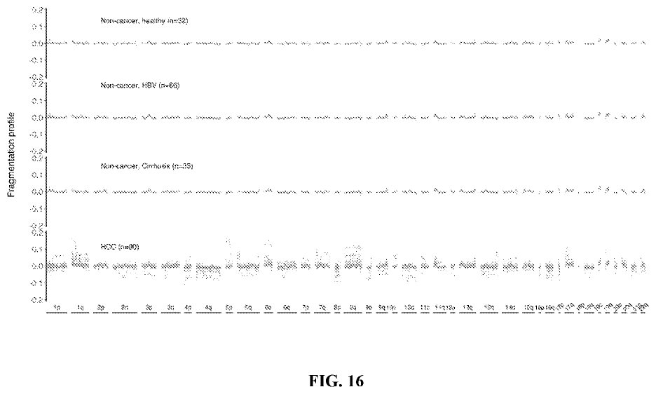

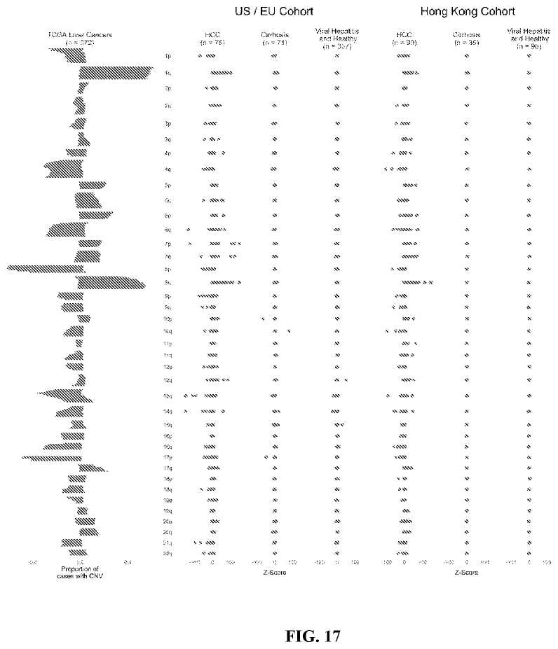

In addition to our cross-validated analysis of the US/EU cohort, we tested the fixed DELFI model in the 223 patients from the Hong Kong cohort. These included patients who had largely resectable early-stage HCC (n=90, stage A=85, B=5) and 101 with cirrhosis or HBV infection. These samples were sequenced previously using a different sequencer (HiSeq 2000 vs Novaseq; 76 bp vs 100 bp read length), different library preparation, and higher number of PCR cycles (14 vs 4 cycles), but we observed similar genome-wide patterns to our earlier analyses (FIG. 15). The fragmentation profiles of patients with viral hepatitis and cirrhosis, as well as healthy individuals, had highly consistent profiles throughout the genome while those of HCC patients were variable and disordered (FIG. 16). Additionally, the chromosomal changes observed in plasma in the Hong Kong cohort were similar to those in the initial US/EU cohort, as well as the cancers from TCGA (FIG. 17). Overall, in this validation cohort, the DELFI model distinguished HCC patients with an AUC of 0.97 from those with high-risk disease (FIG. 4D). These observations suggest that the underlying characteristics of cfDNA fragmentation were similar in this cohort, and that DELFI is a robust method to detect HCC and is generalizable across different high-risk populations.

### Simulation of DELFI Performance at Population Scale

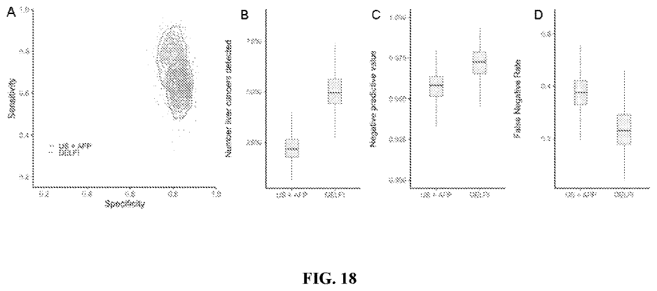

To evaluate how our approach would perform for surveillance and detection in patients at high-risk for liver cancer, we evaluated the DELFI model in a theoretical population of 100,000 high-risk individuals using Monte Carlo simulations. Given the importance of detection of early-stage cancers, we focused our modeling on the detection of stage 0/A disease. We compared the DELFI approach to the current standard of care, concurrent ultrasound and AFP, and modeled the uncertainty of sensitivity and specificity of these surveillance modalities in this theoretical population through probability distributions centered at empirical estimates from our cohort or from previous reports ((11), see Methods). Despite surveillance recommendations, the adherence to HCC surveillance in the US is low, with the most generous estimates suggesting 39% adherence (46), resulting in an average of 40,042 individuals tested in this theoretical population (95% CI, 21,320-61,890). As blood tests offer high accessibility and compliance, with adherence rates of 80-90% reported for blood-based biomarkers (47,48), we conservatively assumed an average of 75% (95% CI, 60-90%) of this population would be tested using the DELFI approach. As the prevalence of cirrhosis, viral hepatitis, and the co-occurrence of these co-morbidities with HCC could vary by region, we used a prior probability distribution to reflect our uncertainty of the composition of these diseases and possible regional differences. Monte Carlo simulations from these probability distributions (Methods) revealed that ultrasound with AFP detected an average of 2,233 (95% CI, 1,088-3,699) individuals with liver cancer (FIG. 18). Using DELFI, we would detect on average 2,794 additional liver cancer cases, or a 2.46-fold increase (95% CI, 1.25-4.57 fold increase) compared to ultrasound with AFP alone (FIG. 18). The DELFI approach would not only substantially improve detection of liver cancer but would be expected to decrease the false negative rate (FNR), or fraction of cancers missed at testing, from 38% for ultrasound with AFP (95% CI, 25%-51.5%) to 24% for DELFI (95% CI, 9%-42.6%). Additionally, the negative predictive value of the test (NPV) would be expected to increase from 95.7% for ultrasound with AFP (95% CI, 93.8%-97.3%) to 97.1% for DELFI (95% CI, 94.8%-99.0%). These analyses suggest a significant population-wide benefit for using a high-specificity blood-based early detection test as a tool for the detection of liver cancer.

### DISCUSSION

Overall, in this study we demonstrate the use of genome-wide cfDNA fragmentome features to detect HCC with high sensitivity and specificity. Furthermore, we show that the fragmentation profiles capture genomic and chromatin characteristics, including alterations known to be important in HCC. Our cfDNA fragmentome approach has robust performance in detecting HCC, including very early-stage disease, independent of disease etiology. To our knowledge, this is the first genome-wide fragmentation analysis that has been independently validated in a separate high-risk population, with stable and robust performance across different racial and ethnic groups from the US and Hong Kong.

Our results also revealed that disease-specific transcription factor signatures can be obtained through analysis of genome-wide cfDNA fragmentation profiles. Although such analyses have been performed using specific transcription factors to distinguish small cell from non-small cell lung cancers (24), this study suggests that analyses of disease-specific transcriptional regulation using genome-wide cfDNA fragmentation may improve the detection and identification of the tissue of origin in patients with cancer. With sufficient numbers of patients, cfDNA transcriptional profiles could further improve machine learning algorithms to detect HCC and other cancers.

HCC is unique in comparison to other solid cancers in that there is a large well-defined high-risk population with an average 3-4% annual risk of developing HCC (49) recommended to have routine cancer screening every six months. Unfortunately, currently available tests have limited diagnostic utility, especially for early stage disease (13). In our study, AFP had 52% sensitivity in detecting HCC, consistent with the known performance of this biomarker (13). Ultrasound-based surveillance also has technical limitations with its operator dependency, and lower sensitivity in obese patients and cirrhosis (50). Most importantly, ultrasound has low compliance to established guidelines, less than 20% worldwide (10,11), compared to much higher adherence of blood tests for other conditions (47). Despite these challenges, HCC screening provides overall survival benefit in patients with HBV (51) and cirrhosis (10), highlighting the major need to improve current screening tests. The high performance of cfDNA fragmentome analyses in HCC detection, along with its cost-efficient characteristics would allow DELFI to be an accessible screening test for HCC and to increase the screening rates beyond the currently dismal levels. An interesting aspect of cfDNA analysis specific to HCC is that transplantation is the most curative treatment for early to intermediate stage HCC, and HCC surveillance of post-transplantation patients with a liquid biopsy approach could have the dual role in tracking recurrence and rejection, as studies of cfDNA in post-transplant patients have shown promise (52).

Although this study represents a potential improvement in current screening approaches, there are some limitations. For example, this study included a relatively small sample size of individuals with HCC. Although the independent validation cohort was performed with pre-analytical differences in laboratory and sequencing methods, the fact that the DELFI approach performed well in this population suggests that the method will ultimately be able to be utilized in a range of different diagnostic laboratories. Larger validation studies will be needed before this approach can be useful clinically. Nevertheless, the observations that scalable and cost-effective non-invasive cfDNA fragmentome analyses can detect liver cancer patients may provide an opportunity to screen high-risk and general populations worldwide.

### Materials and Methods

### Study Population

For the US/EU cohort, samples from 208 patients, including 75 with HCC and 133 high-risk patients without HCC, were collected prospectively as part of HCC biomarker registry and the AIDS Linked to the Intra Venous Experience (ALIVE) study at the Johns Hopkins University School of Medicine, under protocols approved by the Johns Hopkins Institutional Review Board. HCC was defined by histological examination or the appropriate imaging characteristics as defined by accepted guidelines. Tumor staging was determined by the Barcelona Clinic Liver Cancer staging system (BCLC). Detailed clinical data were extracted from the electronic medical record.

High-risk patients were defined as individuals with cirrhosis from any etiology and/or individuals with chronic hepatitis B or hepatitis C who were recommended for routine HCC screening by expert society guidelines (50). In addition, we included 38 patients with Hepatitis B or cirrhosis retrospectively collected by BioIVT (Westbury, NY). AFP levels were quantified by partnering centers in their clinical laboratories using Food and Drug Administration approved AFP tests.

The US/EU cohort also included samples from 293 individuals without cancer that were previously analyzed (24), originally from two screening clinical trial cohorts for colorectal cancer in Denmark (Endoscopy III) and the Netherlands (COCOS, Netherlands Trial Register ID NTR182946). The protocol for the Endoscopy III Project was approved by the Regional Ethics Committee and the Danish Data Protection Agency, and for the COCOS trial, ethical approval was obtained from the Dutch Health Council. The inclusion criteria for both the Dutch and the Danish cohorts were any individuals of age 50-75 eligible for colorectal cancer screening. All patients used had either a FIT negative test or a negative colonoscopy result.

For the Hong Kong cohort, all recruited subjects gave written informed consent, and the study was approved by the Joint Chinese University of Hong Kong and New Territories East Cluster Clinical Research Ethics Committee (15,28).

### Sample Collection and Preservation

The sample collection was performed as follows: venous peripheral blood was collected in one K2-EDTA tube and two serum gel tubes. Within two hours from blood collection tubes were centrifuged at 2330 g at 4° C. for 10 min, plasma was transferred to new tubes and the samples were spun at 14,000 rpm (18,000 rcf) for 10 minutes at room temperature to pellet any remaining cellular debris. After centrifugation EDTA plasma was aliquoted and stored at −80° C. for cfDNA analyses.

### Sequencing Library Preparation

Circulating cell-free DNA was isolated from 2-4 ml of plasma using the Qiagen QIAamp Circulating Nucleic Acids Kit (Qiagen GmbH), eluted in 52 μl of RNase-free water containing 0.04% sodium azide (Qiagen GmbH), and stored in LoBind tubes (Eppendorf AG) at −20° C. Concentration and quality of cfDNA were assessed using the Bioanalyzer 2100 (Agilent Technologies).

Next-generation sequencing (NGS) cfDNA libraries were prepared for WGS using 15 ng cfDNA when available, or entire purified amount when less than 15 ng. In brief, genomic libraries were prepared using the NEBNext DNA Library Prep Kit for Illumina (New England Biolabs (NEB)) with four main modifications to the manufacturer's guidelines: (i) the library purification steps followed the on-bead AMPure XP (Beckman Coulter) approach to minimize sample loss during elution and tube transfer steps; (ii) NEBNext End Repair, A-tailing and adaptor ligation enzyme and buffer volumes were adjusted as appropriate to accommodate on-bead AMPure XP purification; (iii) Illumina dual index adaptors were used in the ligation reaction; and (iv) cfDNA libraries were amplified with Phusion Hot Start Polymerase. All samples underwent a 4 cycle PCR amplification after the DNA ligation step.

### Low Coverage Whole-Genome Sequencing and Alignment

Whole-genome libraries of cancer patients and cancer-free individuals were prepared as in (24) with the modification that they were sequenced using 100-bp paired-end runs (200 cycles) on the Illumina Novaseq platform at 1-2× coverage per genome. Prior to alignment, adapter sequences were filtered from reads using the fastp software (53). Sequence reads were aligned against the hg19 human reference genome using Bowtie2 (54) and duplicate reads were removed using Sambamba (55). Post-alignment, each aligned pair was converted to a genomic interval representing the sequenced DNA fragment using bedtools (56). Only reads with a MAPQ score of at least 30 or greater were retained. Read pairs were further filtered if overlapping the Duke Excluded Regions blacklist (genome.ucsc.edu/cgi-bin/hgTrackUi?db=hg19&g-wgEncodeMapability). To capture large-scale epigenetic differences in fragmentation across the genome estimable from low-coverage WGS, we tiled the hg19 reference genome into non-overlapping 5 Mb bins. Bins with an average GC content <0.3 and an average mappability <0.9 were excluded, leaving 473 bins spanning approximately 2.4 GB of the genome. Following Mathios et al. (24), GC correction was performed independently for short (<150 bp) and long (>=150 bp) cfDNA fragments using an external panel of 20 individuals without cancer sequenced on a NovaSeq to generate a target distribution.

Fastq files for patients in the Hong Kong cohort were obtained from The Chinese University of Hong Kong (CUHK) Circulating Nucleic Acids Research Group, as reported (15, Jiang, 2018 #1645) and processed as described above and in Mathios et. al, to generate the DELFI features. GC correction was performed by normalizing to the target distribution provided in github.com/cancer-genomics/PlasmaToolsNovaseq.hg19, the same target distribution used for GC correction in the US/EU cohort. The validation set consisted of libraries constructed with 14 cycles of PCR and sequenced on the HiSeq 2000. These libraries were normalized to the 4-cycle NovaSeq target distribution to facilitate comparisons between studies. One sample each from the cirrhotic and HBV group were excluded as they were identified to have an HCC diagnosis.

### Chromatin Structure Analysis

A/B compartments for liver cancer tissue and lymphoblastoid cells were obtained from github.com/Jfortin1/TCGA_AB_Compartments as well as from github.com/Jfortin1/HiC_AB_Compartments as described in (29). The two reference tracks were compared to identify informative 100 kb bins, defined as bins where the chromatin domain differed between the two reference tracks or the magnitude difference in eigenvalues that corresponded to a z-score greater than 1.96 or less than −1.96 (p=0.05) across all eigenvalue differences.

The median fragmentation profile for the 10 liver samples of the highest estimated tumor fraction by ichorCNA (57) and 10 randomly selected individuals without cancer was calculated. This information was used to extract an estimated median liver component in the plasma weighted by the ichor score of the individual plasma samples.

### Genome-Wide Transcription Factor Analyses

Chromatin immunoprecipitation followed by sequencing (ChIP-Seq) peaks from 5620 experiments were downloaded from the ReMap 2020 database (33). This set was filtered for experiments with more than 4000 peaks, resulting in 4293 experiments. For each peak in the autosomes we defined the center of the peak as position 0.

The mean of the coverages at each position (−3,000 to +3,000 with respect to the center of each peak) was computed across all peaks for each sample. For the ROC curves, relative coverage was computed for each sample as the mean coverage in a +/−100 bp window surrounding the center of the binding sites divided by the mean coverage in a +/−250 bp window surrounding 2,750 bp upstream and downstream of the binding sites. The ROC curve was generated using pROC 1.16.2 (58). The AUC for each peak set was ranked. Each transcription factor was matched with its NCBI ID, leaving 797 unique transcription factors ranked by AUC. This ranked list was the input for the gseDGN function from the DOSE package in R. The output from this was ranked by the normalized enrichment score (NSE).

### Whole-Genome Fragment Features

Fragmentation features were calculated as in Mathios et. al. (24). Briefly, the ratio of short to long fragments were calculated for 473 non-overlapping 5 MB bins across the genome and z-scores representing arm gains/losses were calculated for autosomal chromosome arms. The principal components of the ratios representing greater than 90% of variance and the z-scores were used to train machine learning models.

### Machine Learning and Cross-Validation Analyses

Two machine learning models were developed, one for high-risk populations (a GBM using the Mathios et al features), and the second for low-risk general populations (a penalized logistic regression with the Mathios et al features as well as coverage from transcription factor binding sites). These models were trained on the US/EU cohort in Caret with 5-fold cross validation with 10 repeats, and scores for each sample were calculated by the mean across repeats and evaluated using AUC-ROC, as in Mathios et. al. (24). The first model was used the high-risk non-cancer and HCC patients while the second used the non-cancer individuals without liver pathology. The locked high-risk model trained on the US/EU cohort was applied to the Hong Kong cohort to generate cancer predictions on an external validation set.

### TCGA Analysis

Copy number data from the HCC cancer cohort in TCGA (LIHC n=372) were retrieved using the package RTCGA v1.16.0 and were analyzed to determine the frequency of copy number gains and losses in the 473 5 mb bins for this cohort (24). The somatic copy number alteration (SCNA) threshold used in Mathios et al. was used to call gains and losses in the HCC cohorts (24,59).

Association of Clinical Covariates with DELFI Score

Potential associations between clinical covariates (for those patients for whom this information was made available) and the DELFI score were assessed with Spearman's rank correlation coefficient (continuous variables) and Kruskal-Wallis one-way analysis of variance (categorical variables).

### Simulation

Monte Carlo simulations were used to compare the DELFI approach to ultrasound and AFP in a theoretical surveillance population. We used estimated 95% confidence intervals of sensitivity and specificity for DELFI and published 95% confidence intervals for ultrasound with AFP (13). The R package epiR was used to derive prior predictive probability distributions (beta distributions) from these confidence intervals ((60) R package version 2.47, CRAN.R-project.org/package=Epi). Zhao et al (2017) reported that adherence to US and AFP surveillance was 39% (95% CI: 21%-65%). As other noninvasive blood-based tests have a reported adherence of more than 75% (47,48) we assumed that adherence to DELFI would be 60% or greater with probability 0.975 or higher. Using these confidence estimates, epiR was used to derive beta prior predictive distributions for adherence. We simulated multinomial probabilities for prevalence of hepatitis B, cirrhosis, hepatitis B+HCC, cirrhosis+HCC, and hepatitis B+cirrhosis+HCC from a Dirichlet with parameters 230, 680, 60, 23, 7, respectively. For a single Monte Carlo simulation for ultrasound with AFP testing, we
(i) sampled the probability of adherence (n) from the prior predictive distribution,
(ii) simulated the number of 100,000 individuals(S) who participated in surveillance (S~Binomial (η, 100,000)),
(iii) sampled probabilities of co-morbidities (Dirichlet (230, 680, 60, 23, 7)),
(iv) computed the prevalence of HCC(θ),
(v) simulated HCC cases (P~Binomial (θ,S)) and computed the number of individuals without cancer (N=S−P),
(vi) sampled the sensitivity (se) and specificity (sp) from the corresponding prior predictive distributions, and
(vii) sampled the true positives (TP~Binomial(P, se)) and false positives (FP~Binomial (N, 1−sp)).

Given TP and FP, we calculated the NPV as (true negatives)/(true negatives+false negatives), where true negatives=N−FP and false negatives=P−TP. We repeated the above simulation 1000 times, obtaining a distribution of TP, FP and NPV. Using parameters for sensitivity, specificity, and adherence for the DELFI approach, we repeated the same Monte Carlo analysis to allow comparisons between these two surveillance methodologies.

TABLE 1
Patient demographics and clinical information

| | Non-cancer | Cancer | |
|---|---|---|---|
| | individuals | patients | |
| Patient Characteristic | n = 426 | n = 75 | P-value* |
| Age | | | |
| Mean | 57.5 | 64.5 | <0.001 |
| Range | 27-81 | 38-88 | |
| Sex | | | |
| Male | 235 | 63 | <0.001 |
| Female | 191 | 12 | |
| Liver disease | | | |
| None | 293 | | <0.001 |
| Hepatitis B | 26 | 1 | |
| Hepatitis C | 29 | 1 | |
| Cirrhosis | 78 | 69 | <0.001 |
| HCV | 53 | 41 | |
| HBV | 2 | 4 | |
| EtOH | 13 | 12 | |
| NAFLD | 3 | 11 | |
| Child Pugh stage | | | |
| A | 20 | 43 | <0.001 |
| B | 10 | 38 | |
| C | 10 | 5 | |
| Unknown | 38 | | |
| BCLC Stage | | | |
| 0 | | 7 | |
| A | | 17 | |
| B | | 30 | |
| C | | 21 | |
| Previous treatment | | | |
| Yes | | 28 | |
| No | | 47 | |
| Validation Cohort (Hong Kong)^{#} | 133 | 90 | |
| Liver Disease | | | |
| None | 32 | | |
| Cirrhosis (HBV) | 35 | 90 | |
| Active HBV | 66 | | |
| BCLC Stage | | | |
| A | | 85 | |
| B | | 5 | |

*P-values were calculated to compare data from individuals with and without liver cancer for the following variables: mean ages using Student's unpaired 2-tailed t-tests, sex distribution, Cirrosis etiology and Child-Pugh stage using a χ² test.
^{#}Validation cohort data were obtained from Jiang et al., PNAS, 2015.

TABLE 2
Top scoring transcription factors in US/EU cohort samples

| Transcription | | | Cell Type in CHIP- | | |
|---|---|---|---|---|---|
| Factor | Gene Name | AUC | seq Experiment | Gene Function* | Link to HCC |
| ZNF512 | Zinc Finger Protein 512 | 0.836 | K-562 | Unknown | Undescribed |
| ZNF184 | Zinc Finger Protein 184 | 0.826 | K-562 | Unknown | Undescribed |
| PCBP2 | Poly(C) binding protein 2 | 0.791 | Hep-G2 | translational | Overexpression contributes to poor prognosis |
| | | | | coregulator | and enhanced cell growth in HCC {Zhang, 2016} |
| JUN | Jun Proto-Oncogene, AP-1 | 0.785 | Hep-G2 | TF | Promotes HBV-related liver tumorigenesis |
| | Transcription Factor Subunit | | | | {Trierweiler, 2016} |
| PHB2 | Prohibitin 2 | 0.771 | K-562 | transcriptional | Functions in mitophagy of HCC {Xiang, 2021} |
| | | | | co-regulator | |
| ATF2 | Activating Transcription Factor 2 | 0.770 | Hep-G2 | TF, HAT | Mediates suppression of liver tumor |
| ATF7 | Activating Transcription Factor 7 | 0.765 | MCF-7 | TF | Regulates growth of liver cancer {Song, 2019} |
| TEAD4 | TEA Domain Transcription Factor 4 | 0.760 | Hep-G2 | TF | Oncogenic role in HCC{Coto-Llerena, 2021} |
| ARID3A | AT-rich interacting domain 3A | 0.756 | Hep-G2 | TF | Facilitates liver cancer malignancy {Shen, 2022} |
| JUND | JunD Proto-Oncogene, AP-1 | 0.754 | HT29_DSMO | TF | PARγ signaling and NAFLD development |
| | Transcription Factor Subunit | | | | {Hasenfusss, 2014} |

TF = Transcription factor,
HAT = Histone acetyl-transferase

### REFERENCES

1. Sung H, Ferlay J, Siegel R L, Laversanne M, Soerjomataram I, Jemal A, et al. Global cancer statistics 2020: GLOBOCAN estimates of incidence and mortality worldwide for 36 cancers in 185 countries. C A: a cancer journal for clinicians 2021; 71(3):209-49.
2. Siegel R L, Miller K D, Fuchs H E, Jemal A. Cancer statistics, 2021. C A: a cancer journal for clinicians 2021; 71(1):7-33.
3. Di Bisceglie A M. Hepatitis C and hepatocellular carcinoma. Hepatology 1997; 26(3 Suppl 1):34S-8S doi 10.1002/hep.510260706.
4. Pinyopornpanish K, Khoudari G, Saleh M A, Angkurawaranon C, Pinyopornpanish K, Mansoor E, et al. Hepatocellular carcinoma in nonalcoholic fatty liver disease with or without cirrhosis: a population-based study. BMC gastroenterology 2021; 21(1):1-7.
5. Donato F, Tagger A, Gelatti U, Parrinello G, Boffetta P, Albertini A, et al. Alcohol and hepatocellular carcinoma: the effect of lifetime intake and hepatitis virus infections in men and women. Am J Epidemiol 2002; 155(4):323-31 doi 10.1093/aje/155.4.323.
6. Waly Raphael S, Yangde Z, Yuxiang C. Hepatocellular carcinoma: focus on different aspects of management. ISRN Oncol 2012; 2012:421673 doi 10.5402/2012/421673.
7. Asrani S K, Devarbhavi H, Eaton J, Kamath P S. Burden of liver diseases in the world. J Hepatol 2019; 70(1):151-71 doi 10.1016/j.jhep.2018.09.014.
8. Frenette C T, Isaacson A J, Bargellini I, Saab S, Singal A G. A Practical Guideline for Hepatocellular Carcinoma Screening in Patients at Risk. Mayo Clin Proc Innov Qual Outcomes 2019; 3(3):302-10 doi 10.1016/j.mayocpiqo.2019.04.005.
9. Kanwal F, Singal A G. Surveillance for hepatocellular carcinoma: current best practice and future direction. Gastroenterology 2019; 157(1):54-64.
10. Singal A G, Pillai A, Tiro J. Early detection, curative treatment, and survival rates for hepatocellular carcinoma surveillance in patients with cirrhosis: a meta-analysis. PLoS medicine 2014; 11(4):e1001624.
11. Singal A G, Yopp A, Skinner C S, Packer M, Lee W M, Tiro J A. Utilization of hepatocellular carcinoma surveillance among American patients: a systematic review. Journal of general internal medicine 2012; 27(7):861-7.
12. Singal A G, Li X, Tiro J, Kandunoori P, Adams-Huet B, Nehra M S, et al. Racial, social, and clinical determinants of hepatocellular carcinoma surveillance. The American journal of medicine 2015; 128(1):90.e1-.e7.
13. Tzartzeva K, Obi J, Rich N E, Parikh N D, Marrero J A, Yopp A, et al. Surveillance imaging and alpha fetoprotein for early detection of hepatocellular carcinoma in patients with cirrhosis: a meta-analysis. Gastroenterology 2018; 154(6):1706-18. e1.
14. Benesova L, Belsanova B, Suchanek S, Kopeckova M, Minarikova P, Lipska L, et al. Mutation-based detection and monitoring of cell-free tumor DNA in peripheral blood of cancer patients. Analytical biochemistry 2013; 433(2):227-34.
15. Jiang P, Chan C W, Chan K C, Cheng S H, Wong J, Wong V W, et al. Lengthening and shortening of plasma DNA in hepatocellular carcinoma patients. Proc Natl Acad Sci USA 2015; 112(11): E1317-25 doi 10.1073/pnas.1500076112.
16. Cai J, Chen L, Zhang Z, Zhang X, Lu X, Liu W, et al. Genome-wide mapping of 5-hydroxymethylcytosines in circulating cell-free DNA as a non-invasive approach for early detection of hepatocellular carcinoma. Gut 2019; 68(12):2195-205 doi 10.1136/gutjnl-2019-318882.
17. Xu R H, Wei W, Krawczyk M, Wang W, Luo H, Flagg K, et al. Circulating tumour DNA methylation markers for diagnosis and prognosis of hepatocellular carcinoma. Nat Mater 2017; 16(11):1155-61 doi 10.1038/nmat4997.
18. Wang Y, Zhou K, Wang X, Liu Y, Guo D, Bian Z, et al. Multiple-level copy number variations in cell-free DNA for prognostic prediction of HCC with radical treatments. Cancer Sci 2021; 112(11):4772-84 doi 10.1111/cas.15128.
19. Kisiel J B, Dukek B A, R VSRK, Ghoz H M, Yab T C, Berger C K, et al. Hepatocellular Carcinoma Detection by Plasma Methylated DNA: Discovery, Phase I Pilot, and Phase II Clinical Validation. Hepatology 2019; 69(3):1180-92 doi 10.1002/hep.30244.
20. Chalasani N P, Ramasubramanian T S, Bhattacharya A, Olson M C, Edwards V D, Roberts L R, et al. A Novel Blood-Based Panel of Methylated DNA and Protein Markers for Detection of Early-Stage Hepatocellular Carcinoma. Clin Gastroenterol Hepatol 2021; 19(12):2597-605 e4 doi 10.1016/j.cgh.2020.08.065.
21. Klein E, Richards D, Cohn A, Tummala M, Lapham R, Cosgrove D, et al. Clinical validation of a targeted methylation-based multi-cancer early detection test using an independent validation set. Annals of Oncology 2021; 32(9):1167-77.
22. Cancer Screening Cost with Galleri. <https:www.galleri.com/the-galleri-test/cost>.
23. Chalasani N P, Porter K, Bhattacharya A, Book A J, Neis B M, Xiong K M, et al. Validation of a Novel Multitarget Blood Test Shows High Sensitivity to Detect Early Stage Hepatocellular Carcinoma. Clin Gastroenterol H 2022; 20(1):173-82. e7.
24. Mathios D, Johansen J S, Cristiano S, Medina J E, Phallen J, Larsen K R, et al. Detection and characterization of lung cancer using cell-free DNA fragmentomes. Nat Commun 2021; 12(1):5060 doi 10.1038/s41467-021-24994-w.
25. Cristiano S, Leal A, Phallen J, Fiksel J, Adleff V, Bruhm D C, et al. Genome-wide cell-free DNA fragmentation in patients with cancer. Nature 2019; 570(7761):385-9 doi 10.1038/s41586-019-1272-6.
26. Zhang X, Wang Z, Tang W, Wang X, Liu R, Bao H, et al. Ultra-Sensitive and Affordable Assay for Early Detection of Primary Liver Cancer Using Plasma cfDNA Fragmentomics. Hepatology 2021.
27. Chen L, Abou-Alfa G K, Zheng B, Liu J F, Bai J, Du L T, et al. Genome-scale profiling of circulating cell-free DNA signatures for early detection of hepatocellular carcinoma in cirrhotic patients. Cell Res 2021; 31(5):589-92 doi 10.1038/s41422-020-00457-7.
28. Jiang P, Sun K, Tong Y K, Cheng S H, Cheng T H T, Heung M M S, et al. Preferred end coordinates and somatic variants as signatures of circulating tumor DNA associated with hepatocellular carcinoma. P Natl Acad Sci USA 2018; 115(46): E10925-E33 doi 10.1073/pnas.1814616115.
29. Fortin J-P, Hansen K D. Reconstructing A/B compartments as revealed by Hi-C using long-range correlations in epigenetic data. Genome biology 2015; 16(1):1-23.
30. Choi J K, Kim Y J. Intrinsic variability of gene expression encoded in nucleosome positioning sequences. Nat Genet 2009; 41(4):498-503 doi 10.1038/ng.319.
31. Ulz P, Perakis S, Zhou Q, Moser T, Belic J, Lazzeri I, et al. Inference of transcription factor binding from cell-free DNA enables tumor subtype prediction and early detection. Nat Commun 2019; 10(1):4666 doi 10.1038/s41467-019-12714-4.
32. Snyder M W, Kircher M, Hill A J, Daza R M, Shendure J. Cell-free DNA Comprises an In Vivo Nucleosome Footprint that Informs Its Tissues-Of-Origin. Cell 2016; 164(1-2):57-68 doi 10.1016/j.cell.2015.11.050.
33. Cheneby J, Menetrier Z, Mestdagh M, Rosnet T, Douida A, Rhalloussi W, et al. ReMap 2020: a database of regulatory regions from an integrative analysis of Human and Arabidopsis DNA-binding sequencing experiments. Nucleic Acids Res 2020; 48(D1):D180-D8 doi 10.1093/nar/gkz945.
34. Bejjani F, Evanno E, Zibara K, Piechaczyk M, Jariel-Encontre I. The AP-1 transcriptional complex: Local switch or remote command? Biochim Biophys Acta Rev Cancer 2019; 1872(1):11-23 doi 10.1016/j.bbcan.2019.04.003.
35. Gozdecka M, Lyons S, Kondo S, Taylor J, Li Y, Walczynski J, et al. JNK suppresses tumor formation via a gene-expression program mediated by ATF2. Cell Rep 2014; 9(4):1361-74 doi 10.1016/j.celrep.2014.10.043.
36. Yan P, Zhou B, Ma Y, Wang A, Hu X, Luo Y, et al. Tracking the important role of JUNB in hepatocellular carcinoma by single-cell sequencing analysis. Oncol Lett 2020; 19(2):1478-86 doi 10.3892/ol.2019.11235.
37. Coto-Llerena M, Tosti N, Taha-Mehlitz S, Kancherla V, Paradiso V, Gallon J, et al. Transcriptional Enhancer Factor Domain Family member 4 Exerts an Oncogenic Role in Hepatocellular Carcinoma by Hippo-Independent Regulation of Heat Shock Protein 70 Family Members. Hepatol Commun 2021; 5(4):661-74 doi 10.1002/hep4.1656.
38. Zhang Z, Fang X, Xie G, Zhu J. GATA3 is downregulated in HCC and accelerates HCC aggressiveness by transcriptionally inhibiting slug expression Corrigendum in/10.3892/ol. 2021.12836. Oncology letters 2021; 21(3):1-.
39. Zhang X, Hua L, Yan D, Zhao F, Liu J, Zhou H, et al. Overexpression of PCBP2 contributes to poor prognosis and enhanced cell growth in human hepatocellular carcinoma. Oncol Rep 2016; 36(6):3456-64.
40. Xiang X, Fu Y, Zhao K, Miao R, Zhang X, Ma X, et al. Cellular senescence in hepatocellular carcinoma induced by a long non-coding RNA-encoded peptide PINT87aa by blocking FOXM1-mediated PHB2. Theranostics 2021; 11(10):4929-44 doi 10.7150/thno.55672.
41. Shen M, Li S, Zhao Y, Liu Y, Liu Z, Huan L, et al. Hepatic ARID3A facilitates liver cancer malignancy by cooperating with CEP131 to regulate an embryonic stem cell-like gene signature. Cell Death & Disease 2022; 13(8):1-13.
42. Marchio A, Pineau P, Meddeb M, Terris B, Tiollais P, Bernheim A, et al. Distinct chromosomal abnormality pattern in primary liver cancer of non-B, non-C patients. Oncogene 2000; 19(33):3733-8 doi 10.1038/sj.one.1203713.
43. Longerich T, Mueller M M, Breuhahn K, Schirmacher P, Benner A, Heiss C. Oncogenetic tree modeling of human hepatocarcinogenesis. Int J Cancer 2012; 130(3):575-83.
44. Stewart S L, Kwong S L, Bowlus C L, Nguyen T T, Maxwell A E, Bastani R, et al. Racial/ethnic disparities in hepatocellular carcinoma treatment and survival in California, 1988-2012. World journal of gastroenterology 2016; 22(38):8584-95 doi 10.3748/wjg.v22.i38.8584.
45. Gupta S, Bent S, Kohlwes J. Test characteristics of alpha-fetoprotein for detecting hepatocellular carcinoma in patients with hepatitis C. A systematic review and critical analysis. Ann Intern Med 2003; 139(1):46-50 doi 10.7326/0003-4819-139-1-200307010-00012.
46. Zhao C, Jin M, Le R H, Le M H, Chen V L, Jin M, et al. Poor adherence to hepatocellular carcinoma surveillance: a systematic review and meta-analysis of a complex issue. Liver International 2018; 38(3):503-14.
47. Bokhorst L P, Alberts A R, Rannikko A, Valdagni R, Pickles T, Kakehi Y, et al. Compliance Rates with the Prostate Cancer Research International Active Surveillance (PRIAS) Protocol and Disease Reclassification in Noncompliers. Eur Urol 2015; 68(5):814-21 doi 10.1016/j.eururo.2015.06.012.
48. Duffy M J, van Rossum L G, van Turenhout S T, Malminiemi O, Sturgeon C, Lamerz R, et al. Use of faecal markers in screening for colorectal neoplasia: a European group on tumor markers position paper. Int J Cancer 2011; 128(1):3-11.
49. Singal A G, Lampertico P, Nahon P. Epidemiology and surveillance for hepatocellular carcinoma: New trends. J Hepatol 2020; 72(2):250-61.
50. Heimbach J K, Kulik L M, Finn R S, Sirlin C B, Abecassis M M, Roberts L R, et al. AASLD guidelines for the treatment of hepatocellular carcinoma. Hepatology 2018; 67(1):358-80 doi 10.1002/hep.29086.
51. Zhang B-H, Yang B-H, Tang Z-Y. Randomized controlled trial of screening for hepatocellular carcinoma. Journal of cancer research and clinical oncology 2004; 130(7):417-22.
52. Goh S K, Do H, Testro A, Pavlovic J, Vago A, Lokan J, et al. The Measurement of Donor-Specific Cell-Free DNA Identifies Recipients With Biopsy-Proven Acute Rejection Requiring Treatment After Liver Transplantation. Transplant Direct 2019; 5(7): e462 doi 10.1097/txd.0000000000000902.
53. Chen S, Zhou Y, Chen Y, Gu J. fastp: an ultra-fast all-in-one FASTQ preprocessor. Bioinformatics 2018; 34(17):1884-i90 doi 10.1093/bioinformatics/bty560.
54. Langmead B, Salzberg S L. Fast gapped-read alignment with Bowtie 2. Nat Methods 2012; 9(4):357-9 doi 10.1038/nmeth.1923.
55. Tarasov A, Vilella A J, Cuppen E, Nijman I J, Prins P. Sambamba: fast processing of NGS alignment formats. Bioinformatics 2015; 31(12):2032-4 doi 10.1093/bioinformatics/btv098.
56. Quinlan A R, Hall I M. BEDTools: a flexible suite of utilities for comparing genomic features. Bioinformatics 2010; 26(6):841-2 doi 10.1093/bioinformatics/btq033.
57. Adalsteinsson V A, Ha G, Freeman S S, Choudhury A D, Stover D G, Parsons H A, et al. Scalable whole-exome sequencing of cell-free DNA reveals high concordance with metastatic tumors. Nat Commun 2017; 8(1):1324 doi 10.1038/s41467-017-00965-y.
58. Robin X, Turck N, Hainard A, Tiberti N, Lisacek F, Sanchez J C, et al. pROC: an open-source package for R and S+ to analyze and compare ROC curves. Bmc Bioinformatics 2011; 12:77 doi 10.1186/1471-2105-12-77.
59. Davoli T, Uno H, Wooten E C, Elledge S J. Tumor aneuploidy correlates with markers of immune evasion and with reduced response to immunotherapy. Science 2017; 355(6322): eaaf8399 doi 10.1126/science.aaf8399.
60. Bendix Carstensen M, Esa L, Michael H. Epi: A package for statistical analysis in epidemiology. R package version 2016; 20.

### OTHER EMBODIMENTS

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

1. A method of diagnosing liver diseases or disorders in a subject, comprising:
isolating circulating cell free DNA (cfDNA) from a subject;
conducting whole genome sequencing of cfDNA molecules to generate genomic libraries and fragmentation profiles;
comparing the fragmentation profiles to healthy subjects, and
diagnosing whether the subject has a liver disease or disorder.

2. The method of claim 1, wherein the fragmentation profiles are consistent for subjects without cancer.

3. The method of claim 1, wherein the fragmentation profiles are highly variable for subjects with liver cancer.

4. The method of claim 1, further comprising identifying cellular origins of the cfDNA fragmentation profiles.

5. The method of claim 4, wherein identifying the cellular origins of cfDNA fragmentation profiles comprises comparing genome-wide fragmentome profiles to high-throughput sequencing chromosome conformation capture (Hi-C).

6. The method of claim 5, wherein the cellular origins of cfDNA fragmentation profiles of healthy subjects correlate to lymphoblastoid cells as the cellular origin.

7. The method of claim 5, wherein the cellular origins of cfDNA fragmentation profiles of subjects with liver cancer correlate to cfDNA fragmentation profiles of chromatin compartments of peripheral blood cells.

8. The method of claim 1, further comprising determining whether cfDNA fragmentation profiles correlate with altered DNA binding of transcription factors.

9. The method of claim 8, wherein the transcription factor DNA binding sites are determined by calculating aggregate cfDNA coverage across all identified transcription factor DNA binding sites compared to overall adjacent genomic coverage to produce a single metric for each transcription factor per sample.

10. The method of claim 9, wherein the transcription factor DNA binding sites identified in subjects with liver cancer are compared to transcription factor DNA binding sites in healthy subjects.

11. (canceled)

12. The method of claim 1, further comprising determining chromosomal gains or losses in liver cancer subjects as compared to healthy subjects.

13. The method of claim 1, further comprising a machine learning model for determining changes in cfDNA fragmentation profiles that classifies the subject as a cancer patient based on the cfDNA fragmentation profile for the subject.

14. The method of claim 13, wherein the machine learning model generates a score for each subject based on a combination of regional and large-scale fragmentation profiles.

15. The method of claim 14, wherein the generated score is diagnostic of liver cancer stages.

16. The method of claim 1 further comprising administering a cancer treatment to the subject that is diagnosed as having a liver disease or disorder.

17. The method of claim 16, wherein the cancer treatment comprises: surgery, adjuvant chemotherapy, neoadjuvant chemotherapy, radiation therapy, hormone therapy, cytotoxic therapy, immunotherapy, adoptive T cell therapy, targeted therapy, and combinations thereof.

18. A method of diagnosing liver cancer, comprising:
isolating circulating cell free DNA (cfDNA) from a biological sample and conducting whole genome sequencing of cfDNA molecules to generate genomic libraries and fragmentation profiles;
identifying cellular origins of the cfDNA fragmentation profiles comprising comparing genome-wide fragmentome profiles to high-throughput sequencing chromosome conformation capture (Hi-C);
correlating cfDNA fragmentation profiles with altered DNA binding of transcription factors;
determining chromosomal gains or losses in liver cancer subjects as compared to healthy subjects;
executing a machine learning model for determining changes in cfDNA fragmentation profiles that classifies the subject as a cancer patient based on the cfDNA fragmentation profile for the subject; thereby,
diagnosing liver cancer in the subject and administering a cancer treatment to the diagnosed subject.

19. (canceled)

20. The method of claim 18, wherein the cellular origins of cfDNA fragmentation profiles of subjects with liver cancer correlate to cfDNA fragmentation profiles of chromatin compartments of peripheral blood cells.

21. The method of claim 18, wherein the transcription factor DNA binding sites are determined by calculating aggregate cfDNA coverage across all identified transcription factor DNA binding sites compared to overall adjacent genomic coverage to produce a single metric for each transcription factor per sample.

22-23. (canceled)

24. The method of claim 18, wherein the machine learning model generates a score for each subject based on a combination of regional and large-scale fragmentation profiles.

25-28. (canceled)
